(19) United States
(12) Patent Application Publication
(10) Pub. No.: US 2020/0179274 A1
(43) Pub. Date: Jun. 11, 2020

(54) DRUG DELIVERY SYSTEM FOR ULTRA-LOW DOSE ESTROGEN COMBINATIONS AND METHODS AND USES THEREOF

(71) Applicant: Lupin Inc., Baltimore, MD (US)

(72) Inventors: Vinita Gupta, Naples, FL (US); Richard Holl, Somerset, NJ (US); James Garegnani, Somerset, NJ (US); Gregory Kaufman, Somerset, NJ (US)

(73) Assignee: Lupin Inc., Baltimore, MD (US)

(21) Appl. No.: 16/695,888

(22) Filed: Nov. 26, 2019

Related U.S. Application Data
(60) Provisional application No. 62/778,090, filed on Dec. 11, 2018.

(51) Int. Cl. A61K 9/00; A61K 31/565; A61K 31/585; A61K 31/567

(52) U.S. Cl. CPC A61K 9/0036; A61K 31/567; A61K 31/585; A61K 31/565

(57) ABSTRACT

The invention relates to a drug delivery system for contraception including a steroidal estrogenic compound, a steroidal progestogenic compound or a combination thereof for the purposes of contraception in a subject, and provides for an ultra-low dose delivery of a steroidal estrogenic compound. The invention also relates to a method of contraception using the drug delivery system and method of manufacturing the drug delivery system.

### CROSS-REFERENCE TO RELATED APPLICATION

This patent application claims priority in and to U.S. Provisional Application No. 62/778,090, filed Dec. 11, 2018, which is hereby incorporated by reference herein in its entirety.

### FIELD OF THE INVENTION

The invention relates to reproductive medicine, for instance, to female contraception. The invention also relates to a drug delivery system for release of a steroidal estrogenic compound, a steroidal progestogenic compound or a combination thereof; a method of manufacturing the drug delivery system; and a method of contraceptive treatment in animals, such as mammals (including, without limitation, humans), using the drug delivery system.

All references and products cited within this application and their disclosures therein are incorporated by reference herein in their entirety.

### BACKGROUND

A variety of chemical and mechanical methods for controlling fertility and for preventing pregnancy are known. Approaches such as sterilization, the use of condoms, intrauterine devices, spermicidal creams and jellies, foam tablets, and oral pills are currently available as a prevention against pregnancy. These methods, though effective to a variable extent, also have limitations. Most of the devices require constant motivation on the part of the user and some approaches such as sterilization and intrauterine devices require specialized medical attention. The oral pill is a popular method of contraception but the oral contraceptives have many undesirable side effects and require the daily ingestion of a tablet. The use of the vaginal ring as a means of administering effective contraceptive steroids through a vaginal route is a means of overcoming some of these drawbacks. For vaginal rings, the concentration of contraceptive agents included in the vaginal rings may pose problems for patients due to adverse side effects associated with high concentration of contraceptive agents.

Estrogen and progestin are molecular classes of two female sex hormones that are used for contraceptive purposes. Combinations of estrogen and progestin work by establishing a contraceptive effect, such as, for example, preventing ovulation (the release of eggs from the ovaries) in a subject; changing the subject's lining of the uterus (womb) to prevent pregnancy from developing; changing the subject's mucus at the cervix (opening of the uterus) to prevent sperm (male reproductive cells) from entering; and/or any other medical condition(s) in the subject that prevents pregnancy.

Combined estrogen-progestin oral contraceptives provide reliable contraception as well as several non-contraceptive benefits. Estrogen- and progestin-based contraceptives, however, have several well-known side effects. Adverse side effects include breast tenderness, nausea, bloating, unscheduled bleeding, weight gain, increased cardiovascular risks, such as, myocardial infarction, hypertension, stroke, and increased risk of venous thromboembolic events.

There have been many attempts to develop low-dose and ultra-low dose estrogen combination oral contraceptives using ethinyl estradiol, a potent estrogen, to reduce the side effects of the estrogen but oral contraceptive have limitations due to adverse side effects and the need to regularly (typically daily) ingest the contraceptive. Current low-dose combination oral contraceptives are defined as orally-administered drug products that contain 35 micrograms or less of ethinyl estradiol intended for once per day administration. Examples of approved and marketed ethinyl estradiol combination products include Yasmin® (drospirenone/ethinyl estradiol tablets) marketed by Bayer, Levora® (levonorgestrel/ethinyl estradiol tablets) marketed by Mayne Pharma, and Ortho-Tri-Cyclen® Lo (norgestimate/ethinyl estradiol tablets) marketed by Janssen.

Other current ultra-low dose combination oral contraceptives are defined as orally-administered drug products that contain 20 micrograms or less of ethinyl estradiol intended for once per day administration. Examples of approved and marketed combination oral contraceptive include Yaz® (drospirenone/ethinyl estradiol tablets) marketed by Bayer and Lo Loestrin® Fe (norethindrone acetate/ethinyl estradiol) marketed by Allergan.

An alternative to oral contraceptives is a vaginal drug delivery system. Such systems, for example, are described in U.S. Pat. Nos. 3,995,633 and 3,995,634, where separate, preferably spherical or cylindrical, reservoirs containing different active substances are assembled in specially constructed holders. A vaginal drug delivery system is also described in U.S. Pat. No. 4,237,885, where a tube or coil of polymeric material is divided into portions by means of a plurality of “spacers” provided within the tube. Each of the separate tube portions is filled with a different active substance in a silicone fluid and the two ends of the tube are subsequently connected to one another. In this vaginal drug delivery system, however, transport (diffusion) of active material from one reservoir to the other takes place through the wall of the tube, especially upon prolonged storage, such that the pre-set fixed release ratio between the active substances in question changes or fluctuates for a period of time. This diffusion can have unintended consequences on a patient's health, and creates an unpredictable system with decreased efficacy and a short storage period.

A two-layered vaginal ring system is described in European Patent Publication No. 0050867. In this system, the ring comprises a pharmacologically acceptable supporting ring covered by two layers preferably of silicone elastomers where the inner layer is a silicone elastomer loaded with an active substance. A similar ring shaped vaginal delivery system is described in U.S. Pat. No. 4,292,965.

In U.S. Pat. No. 4,596,576, a two-compartment vaginal ring is disclosed, where each compartment contains a different active substance. To achieve a suitable ring with a constant release ratio between the various active substances, it was necessary, however, to join the end portions of the compartments by inert stoppers, preferably glass stoppers which can pose a problem during administration of the ring to a subject and in instances where the ring is retained within the subject's vaginal tract.

PCT International Patent Publication No. WO 97/02015 discloses a two-compartment device including a first compartment consisting of a core, a medicated middle layer, and a non-medicated outer layer and a second compartment consisting of a medicated core and a non-medicated outer layer. This structure, however, is complicated and difficult to manufacture.

Another contraceptive intravaginal ring that is currently approved and marketed in the United States is NuvaRing®. NuvaRing® contains 11.7 mg of etonogestrel and 2.7 mg of ethinyl estradiol. After administration, NuvaRing® appears to release about 120 micrograms per day of etonogestrel and about 15 micrograms per day of ethinyl estradiol for a period of 3 weeks (or 21 days). U.S. Pat. No. 5,989,581 discloses a fixed dose combination of progestin compound (like norethindrone) and estrogen compound (like ethinyl estradiol).

A contraceptive vaginal ring releasing norethindrone acetate and ethinyl estradiol was disclosed in a 1994 journal article that includes a core design contraceptive vaginal ring releasing 650 micrograms of norethindrone acetate and 10, 20, 30 or 65 micrograms of ethinyl estradiol daily (Ballagh et al., “A Contraceptive Vaginal Ring Releasing Norethindrone Acetate and Ethinyl Estradiol,” Contraception, 50(6):517-533 (1994)). This ring was developed and tested in 99 women. Two combined contraceptive vaginal rings each releasing approximately 1 mg norethindrone acetate and either 20 micrograms or 15 micrograms of ethinyl estradiol for 24 hours were tested at three clinic sites (Wiseberg et al., “A Comparative Study of Two Contraceptive Vaginal Rings Releasing Norethindrone Acetate and Differing Doses of Ethinyl Estradiol,” Contraception, 59(5):305-310 (1999)).

United States Food and Drug Administration (FDA) has recently approved a contraceptive vaginal ring product, namely, Annovera®. Annovera® is a silicone elastomer vaginal system containing 103 milligrams (mg) of segesterone acetate and 17.4 mg of ethinyl estradiol. Annovera® releases on average 0.15 mg/day of segesterone acetate and 0.013 mg/day of ethinyl estradiol.

One oral combination contraceptive containing estradiol (as the hemihydrate) is approved and marketed in the European Union as Zoely®. Estradiol, however, has low oral bioavailability (2% to 10%) due to first-pass metabolism. Zoely® contains a fixed dose combination of 2.5 mg of nomegestrol acetate and 1.5 mg of estradiol (as the hemihydrate). For Zoely® contraceptive efficacy was achieved with average serum estradiol concentrations of 50 picograms/ml (pg/ml) (see Zoely: EPAR—Product Information—Annex 1 Summary of Product Characteristics for EU dossier).

Another product on the market containing estradiol is Estring®. Estring® is an intravaginal ring that contains 2 mg of estradiol and indicated for the treatment of moderate to severe symptoms of vulvar and vaginal atrophy due to menopause. After administration, Estring® releases around 7.5 microgram of estradiol per day for a period of 90 days. The mean steady state serum estradiol concentration upon administration of Estring® estimates of 7.8, 7.0, 7.0, and 8.1 picograms/mL at weeks 12, 24, 36, and 48, respectively.

Accordingly, there remains a pending need in the medical and pharmaceutical arts to develop a drug delivery system in the form of intravaginal ring that delivers an ultra-low dose of at least one estrogenic compound to reduce side effects in females without affecting its efficacy. Further, the use of bioidentical estrogens in a combination contraceptive product may result in less adverse side-effects.

The invention is directed to overcoming these and other deficiencies in the art.

### SUMMARY

In consideration of the above-identified problems, one aspect of the invention is related to a drug delivery system for use in contraception. The drug delivery system is administered to a subject's vaginal tract to achieve contraception and the system includes a core comprising a steroidal estrogenic compound and a steroidal progestogenic compound. The core of the system is, optionally, covered by a membrane or a skin. The drug delivery system is such that when it is placed in a subject's vaginal tract, it delivers a therapeutically effective dose steroidal estrogenic compound and a therapeutically effective dose of the steroidal progestogenic compound to the subject (optionally, at a controlled rate) for a period of time (or total period of time) to produce a contraceptive effect in the subject. In one embodiment, the therapeutically effective dose of steroidal estrogenic compound is an ultra-low dose.

Another aspect of the invention is related to a method of contraception. This method includes a step of retainably positioning the drug-delivery system within a subject's vaginal tract; retaining the system within the subject's vaginal tract for a period of time; and removing the system from the subject after the period of time. In another embodiment, a method of contraception for a total period of time, which includes (a) retainably positioning the drug-delivery system disclosed in this application within a subject's vaginal tract and retaining the system within the subject's vaginal tract for a first period of time; (b) removing the system from the subject's vaginal tract after the first period of time; (c) reinserting the system within the subject's vaginal tract after a second period of time and retaining the system within the subject's vaginal tract for a third period of time; (d) removing the system from the subject's vaginal tract after the third period of time; (e) optionally reinserting the system within the subject's vaginal tract after the third period of time, retaining the system within the subject's vaginal tract for a fourth period of time, and removing the system from the subject's vaginal tract after the fourth period of time; and (f) optionally repeating step (e) until the end of the total period of time. In another embodiment, the first period of time and third period of time are the same amount of time or different amounts of time. In another embodiment, the second period of time and fourth period of time are the same amount of time or different amounts of time.

Yet another aspect of the invention, a method of manufacturing the drug delivery system disclosed in this application, the method of manufacturing comprising (a) producing the core comprising the combination of the steroidal estrogenic compound and the steroidal progestogenic compound, and (b) covering a portion or all of the core with the membrane to form the drug delivery system. In another aspect of the invention, the core is coextruded with the membrane to form the drug delivery system.

Yet another aspect of the invention is related to a method of making the drug delivery system disclosed in this application. This method includes a step of producing a core comprising the combination of a steroidal estrogenic compound and a steroidal progestogenic compound and a subsequent step of covering a portion or all of the core with a membrane to form the drug delivery system.

In an aspect of the invention, a drug delivery system for use in contraception by administering the system to a subject's vaginal tract, said system comprising (a) a core comprising a steroidal estrogenic compound and a steroidal progestogenic compound; (b) a membrane covering a portion or all of the core; wherein, when the system is in the subject's vaginal tract, the system delivers a therapeutically effective dose of the steroidal estrogenic compound and a therapeutically effective dose of the steroidal progestogenic compound to the subject for a period of time to produce a contraceptive effect in the subject, and the system delivers less than 0.013 mg per day of the steroidal estrogenic compound to the subject's vaginal tract.

In another aspect of the invention, the period of time is about 21 days, about 24 days, about 26 days, or about 3 months after administration to the subject's vaginal tract.

In another aspect of the invention, the drug delivery system delivers the therapeutically effective dose of the steroidal estrogenic compound and the therapeutically effective dose of steroidal progestogenic compound to the subject at a controlled rate for the period of time, and the period of time is about 21 days, about 24 days, about 26 days, or about 3 months after administration to the subject's vaginal tract.

In another aspect of the invention, the steroidal estrogenic compound is ethinyl estradiol or estradiol.

In another aspect of the invention, the drug delivery system delivers about 0.010 mg per day of ethinyl estradiol to the subject for about 21 days, about 24 days, about 26 days, or about 3 months after administration to the subject's vaginal tract.

In another aspect of the invention, the steroidal progestogenic compound is selected from a group consisting of drospirenone, norethindrone, norethindrone acetate, levonorgestrel, etonogestrel, and norgestimate.

In another aspect of the invention, the steroidal progestogenic compound is etonogestrel and the steroidal estrogenic compound is ethinyl estradiol.

In another aspect of the invention, the amount of etonogestrel in the drug delivery system is about 11.7 mg and the amount of ethinyl estradiol in the drug delivery system is about 1.8 mg, or the amount of etonogestrel in the drug delivery system is about 35.1 mg and the amount of ethinyl estradiol in the drug delivery system is about 5.4 mg.

In another aspect of the invention, the drug delivery system releases on an average about 0.12 mg of etonogestrel per day for about 21 days, about 24 days, about 26 days, or about 3 months after administration of the system to the subject's vaginal tract.

In another aspect of the invention, the drug delivery system releases on an average about 0.010 mg of ethinyl estradiol per day for about 21 days, about 24 days, about 26 days, or about 3 months after administration of the system to the subject's vaginal tract.

In another aspect of the invention, the steroidal progestogenic compound is norethindrone acetate and the steroidal estrogenic compound is ethinyl estradiol.

In another aspect of the invention, the amount of norethindrone acetate in the drug delivery system is about 100 mg and the amount of ethinyl estradiol in the drug delivery system is about 1.8 mg, or the amount of norethindrone acetate in the drug delivery system is about 300 mg and the amount of ethinyl estradiol in the drug delivery system is about 5.4 mg.

In another aspect of the invention, the drug delivery system releases on an average about 1 mg of norethindrone acetate per day for about 21 days, about 24 days, about 26 days, or about 3 months after administration of the drug delivery system to the subject's vaginal tract.

In another aspect of the invention, the drug delivery system releases on an average about 0.010 mg of ethinyl estradiol per day for about 21 days, about 24 days, about 26 days, or about 3 months after administration of the drug delivery system to the subject's vaginal tract.

In another aspect of the invention, the steroidal progestogenic compound is etonogestrel and the steroidal estrogenic compound is estradiol.

In another aspect of the invention, the amount of etonogestrel in the drug delivery system is about 11.7 mg and the amount of estradiol in the drug delivery system is about 27 mg, or the amount of etonogestrel in the drug delivery system is about 35.1 mg and the amount of estradiol in the drug delivery system is about 81 mg.

In another aspect of the invention, the drug delivery system releases on an average about 0.12 mg of etonogestrel per day for about 21 days, about 24 days, about 26 days, or about 3 months after administration of the drug delivery system to the subject's vaginal tract.

In another aspect of the invention, the drug delivery system releases on an average about 0.15 mg of estradiol per day for about 21 days, about 24 days, about 26 days, or about 3 months after administration of the drug delivery system to the subject's vaginal tract.

In another aspect of the invention, steroidal progestogenic compound is norethindrone acetate and the steroidal estrogenic compound is estradiol.

In another aspect of the invention, the amount of norethindrone acetate in the drug delivery system is about 100 mg and the amount of estradiol in the drug delivery system is about 27 mg, or the amount of norethindrone acetate in the drug delivery system is about 300 mg and the amount of estradiol in the drug delivery system is about 81 mg.

In another aspect of the invention, the drug delivery system releases on an average about 1 mg of norethindrone acetate per day for about 21 days, about 24 days, about 26 days, or about 3 months after administration of the drug delivery system to the subject's vaginal tract.

In another aspect of the invention, the drug delivery system releases on an average about 0.15 mg of estradiol per day for about 21 days, about 24 days, about 26 days, or about 3 months after administration of the drug delivery system to the subject's vaginal tract.

In another aspect of the invention, the steroidal progestogenic compound is levonorgestrel or drospirenone and the steroidal estrogenic compound is ethinyl estradiol.

In another aspect of the invention, the amount of levonorgestrel in the drug delivery system is about 49 mg and the amount of ethinyl estradiol in the drug delivery system is about 1.8 mg, or the amount of levonorgestrel in the drug delivery system is about 56 mg and the amount of ethinyl estradiol in the drug delivery system is about 2.1 mg, or the amount of levonorgestrel in the drug delivery system is about 60 mg and the amount of ethinyl estradiol in the drug delivery system is about 2.2 mg, or the amount of levonorgestrel in the drug delivery system is about 146 mg and the amount of ethinyl estradiol in the drug delivery system is about 5.4 mg,

In another aspect of the invention, the drug delivery system releases on an average about 0.50 mg of levonorgestrel per day for about 21 days, about 24 days, about 26 days, or about 3 months after administration of the system to the subject's vaginal tract.

In another aspect of the invention, the drug delivery system releases on an average about 0.010 mg of ethinyl estradiol per day for about 21 days, about 24 days, about 26 days, or about 3 months after administration of the system to the subject's vaginal tract.

In another aspect of the invention, the amount of drospirenone in the drug delivery system is about 300 mg and the amount of ethinyl estradiol in the drug delivery system is about 1.8 mg, or the amount of drospirenone in the drug delivery system is about 343 mg and the amount of ethinyl estradiol in the drug delivery system is about 2.1 mg, or the amount of drospirenone in the drug delivery system is about 371 mg and the amount of ethinyl estradiol in the drug delivery system is about 2.2 mg.

In another aspect of the invention, the drug delivery system releases on an average about 3 mg of drospirenone per day for about 21 days or about 24 days after administration of the system to the subject's vaginal tract, or an average about 1 mg of drospirenone per day for about 26 days after administration of the system to the subject's vaginal tract.

In another aspect of the invention, the drug delivery system releases on an average about 0.010 mg of ethinyl estradiol per day for about 21 days, about 24 days, or about 26 days after administration of the system to the subject's vaginal tract.

In another aspect of the invention, the steroidal progestogenic compound is levonorgestrel or drospirenone and the steroidal estrogenic compound is estradiol.

In another aspect of the invention, the amount of levonorgestrel in the drug delivery system is about 49 mg and the amount of estradiol in the drug delivery system is about 27 mg, or the amount of levonorgestrel in the drug delivery system is about 56 mg and the amount of estradiol in the drug delivery system is about 31 mg, or the amount of levonorgestrel in the drug delivery system is about 60 mg and the amount of estradiol in the drug delivery system is about 33 mg, or the amount of levonorgestrel in the drug delivery system is about 146 mg and the amount of estradiol in the drug delivery system is about 81 mg.

In another aspect of the invention, the drug delivery system releases on an average about 0.5 mg of levonorgestrel per day for about 21 days, about 24 days, about 26 days, or about 3 months after administration of the system to the subject's vaginal tract.

In another aspect of the invention, the drug delivery system releases on an average about 0.15 mg of estradiol per day for about 21 days, about 24 days, about 26 days, or about 3 months after administration of the system to the subject's vaginal tract.

In another aspect of the invention, the amount of drospirenone in the drug delivery system is about 300 mg and the amount of estradiol in the drug delivery system is about 27 mg, or the amount of drospirenone in the drug delivery system is about 343 mg and the amount of estradiol in the drug delivery system is about 31 mg, or the amount of drospirenone in the drug delivery system is about 371 mg and the amount of estradiol in the drug delivery system is about 33 mg.

In another aspect of the invention, the drug delivery system releases on an average about 3 mg of drospirenone per day for about 21 days or about 24 days after administration of the system to the subject's vaginal tract, or an average about 1 mg of drospirenone per day for about 26 days after administration of the system to the subject's vaginal tract.

In another aspect of the invention, the drug delivery system releases on an average about 0.15 mg of estradiol per day for about 21 days, about 24 days, or about 26 days after administration of the system to the subject's vaginal tract.

In another aspect of the invention, the core is made of a thermoplastic polymer or an elastomer.

In another aspect of the invention, the core is made of a material selected from the group consisting of low-density polyethylene, ethylene-vinyl acetate copolymers, thermoplastic polyurethane, styrene-butadiene-styrene copolymers, and a combination thereof.

In another aspect of the invention, the membrane is made of a material selected from the group consisting of olefins, vinyl polymers, rubber polymers, silicon polymers, microporous polymers, diffusion polymers, polyethylene, ethylene-vinyl acetate copolymers, thermoplastic polyurethanes, polyether-ester polymers, cellulose, and a combination thereof.

In another aspect of the invention, the drug delivery system is adapted for administration in the subject's vaginal tract.

In another aspect of the invention, the drug delivery system is substantially in the form of a ring, a sphere, a cylinder, an implant, an intrauterine system, a helical coil, a toroid, a spring, or a combination thereof.

In another aspect of the invention, the drug delivery system is substantially in the shape of a ring. In another aspect of the invention, the drug delivery ring has an outer diameter of from about 50 millimeters to about 60 millimeters, or an outer diameter of from about 52 millimeters to about 56 millimeters. In another aspect of the invention, the ring has a cross-sectional diameter of from about 2.5 millimeters to about 5 millimeters.

In another aspect of the invention, the steroidal estrogenic compound and the steroidal progestogenic compound are enclosed in separate sections of the core, or enclosed in the same section of the core. In another aspect of the invention, the steroidal estrogenic compound and the steroidal progestogenic compound are enclosed in same or separate compartments within the core. In another aspect of the invention, the core includes a fixed ratio of the steroidal estrogenic compound to the steroidal progestogenic compound.

In another aspect of the invention, the subject is a mammal or human.

### BRIEF DESCRIPTION OF THE DRAWINGS

This description of the exemplary embodiments is intended to be read in connection with the accompanying drawings, which are to be considered part of the entire written description. The features of the embodiments described herein will be more fully disclosed in the following detailed description, which is to be considered together with the accompanying drawings wherein like numbers refer to like parts and further wherein. The drawing figures are not necessarily to scale and certain features may be shown exaggerated in scale or in somewhat schematic form in the interest of clarity and conciseness.

FIG. 1 shows a planar view of a drug delivery system (1) in the form of an annular vaginal ring having a ring-shaped core (12) surrounded on all sides by a membrane or a skin (11 and 13) where the membrane is permeable to active agents, such as contraceptive agents, included in the core and controls the rate of release of at least one active agent included in the core. One or more drugs or active agents may be evenly or unevenly dissolved, spread, distributed or included within the core of the ring. The vaginal ring is made of soft, resilient, pliable material such that it may be administered and retained in the vaginal tract of the subject.

FIG. 2 is a planar view of a drug delivery system (2) in the form of an annular vaginal ring having a ring-shaped core (23) surrounded on all sides by a membrane or a skin (21, 24) where the membrane controls the rate of release of at least one drug or active agent (22) included in the core. One or more active agents or drugs (22) are included in the core of the ring in the form of granules, crystals, pellets or other similar structures. The drug or the active agent may be distributed evenly or unevenly within the core of the ring. The vaginal ring is optionally made of soft, resilient, pliable material such that it may be administered and retained in the vaginal tract of the subject.

FIG. 3 is a drug delivery system (3) in the form of multiple annular rings where each ring has a core (31, 32, 33) and the core is optionally surrounded by a membrane or skin. Each of the rings may include the same active agent, a combination of active agents, or each ring may include a different active agent. The rings may be joined together, wrapped together, or concatenated together for easy delivery to a subject. Optionally, each ring may include a membrane or a skin where the membrane controls the rate of release of at least one active agent included within the core of the ring. In one embodiment, at least one ring is made of soft, resilient, pliable material such that it may be administered and retained in the vaginal tract of the subject.

FIG. 4 is a drug delivery system (4) in the form of a vaginal ring where the ring includes a retaining ring (42) that is attached to multiple containers (41). The containers, for example, may include a drug or an active agent that is distributed evenly or unevenly within the containers. The container may, in one instance, include one or more drugs or active agents dissolved in a suitable solvent. The ring may optionally be surrounded on all sides by a membrane where the membrane controls the rate of release of at least one drug or active agent included within one or more containers.

FIG. 5 is a drug delivery system (5) in the form of an annular vaginal ring that includes a core (51) where the core includes two compartments (52, 53) embedded within the core. Each compartment includes a drug or an active agent or a combination of drugs and active agents. The ring may optionally be surrounded on all sides by a membrane where the membrane controls the rate of release of at least one drug or active agent included within the compartment.

### DETAILED DESCRIPTION

The invention is not limited to particular drug delivery systems, processes, compounds, or methodologies described, as these may vary. The terminology used in the description is for the purpose of describing particular versions or embodiments only, and is not intended to limit the scope of the invention. Unless defined otherwise, all technical and scientific terms used herein have the same meanings as commonly understood by one of ordinary skill in the art. Any methods and materials similar or equivalent to those described herein can be used in the practice or testing of embodiments of the invention, the preferred methods, devices, and materials described herein.

The active agent, drugs, or therapeutics of the invention include antifertility or contraceptive steroids. These steroids include progestogenic steroids that have antifertility properties and estrogenic steroids that also have antifertility properties. These active agents can be of naturally occurring or synthetic origin.

As referred to herein, the term “steroidal estrogenic compound” includes, but not limited to, estrogen and compounds that imitate/mimic the effects of estrogen in a human or animal subject. They can either be synthetic or natural chemical compounds. Estrogen is the predominant sex hormone in women, affecting the development and function of the female reproductive system, including the endometrium, uterus and mammary glands. Estrogen is commonly used as a contraceptive, and is a major component of menopausal hormone therapy. Estrogens are also used in postmenopausal women to treat urinary stress incontinence, dizziness, fatigue, irritability, and to prevent osteoporosis. Endogenous natural estrogens are C18 steroids and include estrone (E1), estradiol (E2), estriol (E3), and estretrol (E4). Some examples of steroidal estrogenic compounds that may be used in the invention are α-estradiol; α-estradiol-3-benzoate; 17α-cyclopentanepropionate estradiol; 1,3,5,(10)-estratriene-3, 17α-diol dipropionate; estra-1,3,5(10)-triene-3, 17α-diol valerate; estrone; ethinyl estradiol; 17-ethinylestradiol-3-methyl ether; mestranol, 17-ethinyl estradiol-3-cyclopentoether estroil; estradiol (E2); estriol (E3); and estretrol (E4) and mixtures thereof, and the like.

As referred to herein, the term “steroidal progestogenic compound” includes, but not limited to, progestogens, progestins, and compounds that imitate the effects of progestin in a human or animal subject. Progestogens generically describe steroids possessing progestational activity and the term progestins are used to describe synthetic steroids that have progestational effects. Progesterone is an endogenous steroid and progestogen sex hormone involved in the menstrual cycle, pregnancy, and embryogenesis of humans and other species. It belongs to a group of steroid hormones called the progestogens, and is the major progestogen in the body. Progesterone has a variety of important functions in the body. It is also a crucial metabolic intermediate in the production of other endogenous steroids, including the sex hormones and the corticosteroids, and plays an important role in brain function as a neurosteroid.

As used herein, the singular forms “a”, “an” and “the” include plural reference unless the context clearly dictates otherwise.

As used herein, the term “about”, is intended to qualify the numerical values which it modifies, denoting such a value as variable within a margin of error. When no particular margin of error, such as a standard deviation to a mean value, is recited, the term “about” means plus or minus 10% of the numerical value of the number with which it is being used. Therefore, e.g., about 50% means in the range of 45%-55%.

As used herein, the terms “patient” and “subject” are interchangeable and may be taken to mean any living organism that may be treated with drug delivery systems, compounds or compositions of the invention. As such, the terms “patient” and “subject” may include, but are not limited to, any non-human mammal, primate or human. In some embodiments, the “patient” or “subject” is an adult, child, infant, or fetus. In some embodiments, the term “patient” or “subject” is a human. In some embodiments, the term “patient” or “subject” is a mammal, such as mice, rats, other rodents, rabbits, dogs, cats, swine, cattle, sheep, horses, primates, or humans.

In each of the embodiments disclosed herein, the active agents, therapeutics, pharmaceutical compositions, and methods may be utilized with or on a subject in need of such treatment, which may also be referred to as “in need thereof.” As used herein, the phrase “in need thereof” means that the subject has been identified as having a need for the particular method or treatment or that the treatment has been given to the subject for a particular purpose.

The terms “treat,” “treating,” and “treatment” are meant to include alleviating or abrogating a pregnancy, the chances of conception of an offspring, a disease/disorder, or one or more of the symptoms associated with a disease/disorder or alleviating or eradicating the cause(s) of the disease/disorder itself. The terms “treat,” “treating,” and “treatment” also include prevention, for example, prevention of a pregnancy. The terms “prevent,” “preventing,” and “prevention” refer to a method of delaying or precluding the onset of a disease/disorder; and/or its attendant symptoms, barring a subject from acquiring a disease/disorder or reducing a subject's risk of acquiring a disease/disorder; barring a subject from conceiving an offspring and/or preventing a pregnancy.

As used herein, the term “administering” when used in conjunction with a drug delivery system means to administer the delivery system directly or indirectly into or onto a target tissue or to a patient whereby the drug or therapeutic in the delivery system locally or systemically affects the tissue or the patient. “Administering” a drug delivery system or a composition may be accomplished by vaginal administration, oral administration, injection, infusion, inhalation, implantation, absorption or by any method in combination with other known techniques. “Administering” may include the act of self-administration or administration by another person such as by a health care provider.

As used herein, the term “pharmaceutical composition” means a composition including at least one or more active agents or compounds described in the invention. The pharmaceutical composition is amenable to investigation for a specified, efficacious outcome in a mammal (for example, without limitation, a human). Those of ordinary skill in the art will understand and appreciate the techniques appropriate for determining whether the active agents of the pharmaceutical composition have a desired efficacious outcome based upon the needs of the artisan.

The term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, excipient, solvent, or encapsulating material. Each component must be “pharmaceutically acceptable” in the sense of being compatible with the other ingredients of a pharmaceutical formulation. It must also be suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st Edition; Lippincott Williams & Wilkins: Philadelphia, Pa., 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, Fla., 2004). Where relevant, all embodiments of the invention, including the drugs, active agents or compounds disclosed herein, may be formulated such that they include a “pharmaceutically acceptable carrier,” “pharmaceutically acceptable excipient,” “physiologically acceptable carrier,” or “physiologically acceptable excipient.”

The terms “therapeutically effective amount” or “therapeutic dose” are used interchangeably and refer to the amount of an active agent, pharmaceutical compound, or composition that elicits a clinical, biological or medicinal response in a tissue, system, animal, individual, or human that is being sought by a researcher, veterinarian, medical doctor, or other clinical professional. A clinical, biological or medical response may include, for example, one or more of the following: (1) preventing a pregnancy, disease, condition or disorder in an individual, (2) inhibiting a pregnancy, disease, condition or disorder in an individual, and (3) ameliorating a disease, condition or disorder in an individual that is experiencing or exhibiting the pathology or symptoms of the disease, condition or disorder or reversing the pathology and/or symptoms experience or exhibited by the individual.

The term “pharmaceutically acceptable salt” for the purpose of the invention is meant to indicate, without any limitation, those salts which are within the scope of sound medical judgment, suitable for use in contact with the tissues of a patient, without undue toxicity, irritation, allergic response and the like, and are commensurate with a reasonable benefit/risk ratio. Pharmaceutically acceptable salts suitable for the invention are well known in the art and described in, for instance, Berge et al., “Pharmaceutical Salts,” J. Pharm. Sci., 66(1):1-19 (1977).

The phrase “delivery system” as used herein refers to a system manufactured in the form of a delivery device, which provides a pre-programmed, unattended delivery of an agent, optionally, at a controlled rate, and for a period of time (or total period of time) established to meet a specific need.

The phrase “ultra-low dose” when used in the context of drug delivery systems described herein is considered to be those drug delivery systems that release less than 0.013 mg of steroid estrogenic compound per day. For example, an ultra-low dose can be about 0.010 mg per day of ethinyl estradiol or an average about 0.010 mg of ethinyl estradiol per day for about 21 days, about 24 days, about 26 days, or about 3 months.

One aspect of the invention is related to a drug delivery system for use in contraception. The drug delivery system is administered to a subject's vaginal tract to achieve contraception, and the system includes a core comprising a steroidal estrogenic compound and a steroidal progestogenic compound. The core of the system is, optionally, covered by a membrane or a skin. The drug delivery system is such that when it is placed in a subject's vaginal tract, it delivers a therapeutically effective dose steroidal progestogenic compound and a therapeutically effective dose of the steroidal estrogenic compound to the subject at a controlled rate for a period of time to produce a contraceptive effect in the subject. In one embodiment, the therapeutically effective dose of steroidal estrogenic compound is an ultra-low dose.

In one embodiment, the drug delivery system of the invention causes simultaneous release of two or more active agent wherein the active agent can be, e.g., the steroidal estrogenic compound or the steroidal progestogenic compound. In an embodiment, the drug delivery system releases the active agents in a substantially constant ratio for a period of time (or total period of time). In another embodiment, the drug delivery system or the core of the drug delivery system includes a fixed/constant ratio of the steroidal estrogenic compound to the steroidal progestogenic compound for a period of time (or total period of time). The above-mentioned substantially constant ratio and the fixed/constant ratio are examples of a controlled rate. The system may have one or multiple controlled rates for the period of time (or total period of time).

The steroidal estrogenic compound for the purposes of this invention can be selected form a variety of well-known synthetic and naturally occurring estrogenic compounds. For example, the steroidal estrogenic compound can be selected from the group consisting of ethinyl estradiol; estradiol; α-estradiol; α-estradiol-3-benzoate; 17α-cyclopentanepropionate estradiol; 1,3,5,(10)-estratriene-3, 17α-diol dipropionate; estra-1,3,5(10)-triene-3, 17α-diol valerate; 17-ethinyl estradiol-3-methyl ether; mestranol, 17-ethinyl estradiol-3-cyclopentoether estroil; estrone (E1); estradiol (E2); estriol (E3); or estretrol (E4) or mixtures thereof, and the like. In one preferred embodiment, the drug delivery systems of the invention include one or more bioidentical estrogens, such as estrone (E1), estradiol (E2), estriol (E3), or estretrol (E4).

In a preferred embodiment, the steroidal estrogenic compound is ethinyl estradiol. In another preferred embodiment, the steroidal estrogenic compound is estradiol.

The steroidal progestogenic compound for the purposes of this invention can be selected form a variety of well-known synthetic and naturally occurring progestogenic compounds. For example, the steroidal estrogenic compound can be selected from the group consisting of drospirenone, norethindrone, norethindrone acetate, levonorgestrel, etonogestrel, and norgestimate.

In one embodiment, the invention includes a drug delivery system having combination of progestin and ultra-low dose estrogen for the purpose of contraception. In another embodiment, the drug delivery system is such that the steroidal progestogenic compound is etonogestrel and the steroidal estrogenic compound is ethinyl estradiol. In another embodiment, the drug delivery system is such that the steroidal progestogenic compound is norethindrone acetate and the steroidal estrogenic compound is ethinyl estradiol. In another embodiment, the drug delivery system is such that the steroidal progestogenic compound is levonorgestrel and the steroidal estrogenic compound is ethinyl estradiol. In another embodiment, the drug delivery system is such that the steroidal progestogenic compound is drospirenone and the steroidal estrogenic compound is ethinyl estradiol.

In one embodiment, the drug delivery system includes ethinyl estradiol and the system delivers about 0.010 mg per day of ethinyl estradiol to the subject for about 21 days after administration. In one embodiment, the drug delivery system includes ethinyl estradiol and the system delivers about 0.010 mg per day of ethinyl estradiol to the subject for about 24 days after administration. In one embodiment, the drug delivery system includes ethinyl estradiol and the system delivers about 0.010 mg per day of ethinyl estradiol to the subject for about 26 days after administration. In another embodiment, the drug delivery system includes ethinyl estradiol and the system delivers about 0.010 mg per day of ethinyl estradiol to the subject for about 3 months after administration.

The drug delivery systems described herein release the steroidal progestogenic compound, steroidal estrogenic compound, or a combination thereof for a period of time to the subject. In one embodiment, the drug delivery system delivers a therapeutically effective dose of the steroidal progestogenic compound for a period of time wherein the time is about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 15 days, about 20 days, about 21 days, about 24 days, about 25 days, about 26 days, about 30 days, about 35 days, about 40 days, about 45 days, about 50 days, about 55 days, about 60 days, about 65 days, about 70 days, about 75 days, about 80 days, about 85 days, or about 90 days to the subject. In another embodiment, the drug delivery system delivers a therapeutically effective dose of the steroidal estrogenic compound for a period of time wherein the time is about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 15 days, about 20 days, about 21 days, about 24 days, about 25 days, about 26 days, about 30 days, about 35 days, about 40 days, about 45 days, about 50 days, about 55 days, about 60 days, about 65 days, about 70 days, about 75 days, about 80 days, about 85 days, or about 90 days to the subject. In another embodiment, the drug delivery system delivers a therapeutically effective dose of the steroidal estrogenic compound and a therapeutically effective dose of the steroidal progestogenic compound for a period of time wherein the time is about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 15 days, about 20 days, about 21 days, about 24 days, about 25 days, about 26 days, about 30 days, about 35 days, about 40 days, about 45 days, about 50 days, about 55 days, about 60 days, about 65 days, about 70 days, about 75 days, about 80 days, about 85 days, or about 90 days to the subject. In a preferred embodiment, the drug delivery system delivers a therapeutically effective dose of the steroidal progestogenic compound and the steroidal estrogenic compound for a period of about or at least 21 days or up to 3 months to the subject's vaginal tract.

In one embodiment, the drug delivery system includes from about 1 mg etonogestrel to about 75 mg etonogestrel. In one embodiment, the drug delivery system includes about 5, 10, 15, 20, 25, 30, 35, 40, 45, 50, 55, 60, 65, 70, or 75 mg of etonogestrel. In one embodiment, the drug delivery system includes from about 0.1 mg to about 7.5 mg ethinyl estradiol. In another embodiment, the drug delivery system includes about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, or 7.5 mg of ethinyl estradiol. In a preferred embodiment, the drug delivery system includes about 11.7 mg of etonogestrel and about 1.8 mg of ethinyl estradiol. In another preferred embodiment, the drug delivery system includes about 13.4 mg of etonogestrel and about 2.1 mg of ethinyl estradiol. In another preferred embodiment, the drug delivery system includes about 14.5 mg of etonogestrel and about 2.2 mg of ethinyl estradiol. In another preferred embodiment, the drug delivery system includes about 35.1 mg of etonogestrel and about 5.4 mg of ethinyl estradiol. In one embodiment, the drug delivery system releases on an average from about 0.1 mg to about 0.2 mg of etonogestrel per day for about 21 days. In one embodiment, the drug delivery system releases on an average from about 0.1 mg to about 0.2 mg of etonogestrel per day for about 24 days. In one embodiment, the drug delivery system releases on an average from about 0.1 mg to about 0.2 mg of etonogestrel per day for about 26 days. In one embodiment, the drug delivery system releases on an average from about 0.1 mg to about 0.2 mg of etonogestrel per day for about 3 months. In another embodiment, the drug delivery system releases on an average about 0.10, 0.11, 0.12, 0.13, 0.14, 0.15, 0.16, 0.17, 0.18, 0.19 or 0.20 mg of etonogestrel per for day for about 21 days after administration to the subject's vaginal tract. In another embodiment, the drug delivery system releases on an average about 0.10, 0.11, 0.12, 0.13, 0.14, 0.15, 0.16, 0.17, 0.18, 0.19 or 0.20 mg of etonogestrel per for day for about 24 days after administration to the subject's vaginal tract. In another embodiment, the drug delivery system releases on an average about 0.10, 0.11, 0.12, 0.13, 0.14, 0.15, 0.16, 0.17, 0.18, 0.19 or 0.20 mg of etonogestrel per for day for about 26 days after administration to the subject's vaginal tract. In another embodiment, the drug delivery system releases on an average about 0.10, 0.11, 0.12, 0.13, 0.14, 0.15, 0.16, 0.17, 0.18, 0.19 or 0.20 mg of etonogestrel per for day for about 3 months after administration to the subject's vaginal tract. In a preferred embodiment, the system releases on an average about 0.12 mg of etonogestrel per for day for about 21 days after administration to the subject's vaginal tract. In another preferred embodiment, the system releases on an average about 0.12 mg of etonogestrel per for day for about 24 days after administration to the subject's vaginal tract. In another preferred embodiment, the system releases on an average about 0.12 mg of etonogestrel per for day for about 26 days after administration to the subject's vaginal tract. In another preferred embodiment, the system releases on an average about 0.12 mg of etonogestrel per for day for about 3 months after administration to the subject's vaginal tract. In one embodiment, the drug delivery system releases on an average from about 0.001 to about 0.013 mg of ethinyl estradiol per day for about 21 days after administration to the subject's vaginal tract. In one embodiment, the drug delivery system releases on an average from about 0.001 to about 0.013 mg of ethinyl estradiol per day for about 24 days after administration to the subject's vaginal tract. In one embodiment, the drug delivery system releases on an average from about 0.001 to about 0.013 mg of ethinyl estradiol per day for about 26 days after administration to the subject's vaginal tract. In one embodiment, the drug delivery system releases on an average from about 0.001 to about 0.013 mg of ethinyl estradiol per day for about 3 months after administration to the subject's vaginal tract. In another embodiment, the drug delivery system releases on an average about 0.001, 0.002, 0.003, 0.004, 0.005, 0.006, 0.007, 0.008, 0.009, 0.010, 0.012, and 0.013 mg of ethinyl estradiol per for day for about 21 days after administration to the subject's vaginal tract. In another embodiment, the drug delivery system releases on an average about 0.001, 0.002, 0.003, 0.004, 0.005, 0.006, 0.007, 0.008, 0.009, 0.010, 0.012, and 0.013 mg of ethinyl estradiol per for day for about 24 days after administration to the subject's vaginal tract. In another embodiment, the drug delivery system releases on an average about 0.001, 0.002, 0.003, 0.004, 0.005, 0.006, 0.007, 0.008, 0.009, 0.010, 0.012, and 0.013 mg of ethinyl estradiol per for day for about 26 days after administration to the subject's vaginal tract. In another embodiment, the drug delivery system releases on an average about 0.001, 0.002, 0.003, 0.004, 0.005, 0.006, 0.007, 0.008, 0.009, 0.010, 0.012, and 0.013 mg of ethinyl estradiol per for day for about 3 months after administration to the subject's vaginal tract. In a preferred embodiment, the system releases on an average about 0.010 mg of ethinyl estradiol per for day for about 21 days after administration to the subject's vaginal tract. In another preferred embodiment, the system releases on an average about 0.010 mg of ethinyl estradiol per for day for about 24 days after administration to the subject's vaginal tract. In another preferred embodiment, the system releases on an average about 0.010 mg of ethinyl estradiol per for day for about 26 days after administration to the subject's vaginal tract. In another preferred embodiment, the system releases on an average about 0.010 mg of ethinyl estradiol per for day for about 3 months after administration to the subject's vaginal tract.

In one embodiment, the steroidal progestogenic compound in the drug delivery system is norethindrone acetate and the steroidal estrogenic compound in the drug delivery system is ethinyl estradiol. Such drug delivery system includes from about 50 mg to about 750 mg of norethindrone acetate. In another embodiment, the drug delivery system includes about 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, or 750 mg of norethindrone acetate. This drug delivery system includes from about 0.1 mg to about 7.5 mg ethinyl estradiol. In another embodiment, the drug delivery system includes about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, or 7.5 mg of ethinyl estradiol. In a preferred embodiment, the drug delivery system includes about 100 mg of norethindrone acetate and about 1.8 mg of ethinyl estradiol. In another preferred embodiment, the drug delivery system includes about 114 mg of norethindrone acetate and about 2.1 mg of ethinyl estradiol. In another preferred embodiment, the drug delivery system includes about 124 mg of norethindrone acetate and about 2.2 mg of ethinyl estradiol. In another preferred embodiment, the drug delivery system includes about 300 mg of norethindrone acetate and about 5.4 mg of ethinyl estradiol. In one embodiment, the drug delivery system releases on an average from about 0.1 mg to about 2 mg of norethindrone acetate per day for about 21 days. In one embodiment, the drug delivery system releases on an average from about 0.1 mg to about 2 mg of norethindrone acetate per day for about 24 days. In one embodiment, the drug delivery system releases on an average from about 0.1 mg to about 2 mg of norethindrone acetate per day for about 26 days. In one embodiment, the drug delivery system releases on an average from about 0.1 mg to about 2 mg of norethindrone acetate per day for about 3 months. In another embodiment, the drug delivery system releases on an average about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, or 2 mg of norethindrone acetate per for day for about 21 days after administration to the subject's vaginal tract. In another embodiment, the drug delivery system releases on an average about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, or 2 mg of norethindrone acetate per for day for about 24 days after administration to the subject's vaginal tract. In another embodiment, the drug delivery system releases on an average about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, or 2 mg of norethindrone acetate per for day for about 26 days after administration to the subject's vaginal tract. In another embodiment, the drug delivery system releases on an average about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, or 2 mg of norethindrone acetate per for day for about 3 months after administration to the subject's vaginal tract. In a preferred embodiment, the system releases on an average about 1 mg of norethindrone acetate per for day for about 21 days after administration to the subject's vaginal tract. In another preferred embodiment, the system releases on an average about 1 mg of norethindrone acetate per for day for about 24 days after administration to the subject's vaginal tract. In another preferred embodiment, the system releases on an average about 1 mg of norethindrone acetate per for day for about 26 days after administration to the subject's vaginal tract. In another preferred embodiment, the system releases on an average about 1 mg of norethindrone acetate per for day for about 3 months after administration to the subject's vaginal tract. In one embodiment, the drug delivery system releases on an average from about 0.001 to about 0.013 mg of ethinyl estradiol per day for about 21 days after administration to the subject's vaginal tract. In one embodiment, the drug delivery system releases on an average from about 0.001 to about 0.013 mg of ethinyl estradiol per day for about 24 days after administration to the subject's vaginal tract. In one embodiment, the drug delivery system releases on an average from about 0.001 to about 0.013 mg of ethinyl estradiol per day for about 26 days after administration to the subject's vaginal tract. In one embodiment, the drug delivery system releases on an average from about 0.001 to about 0.013 mg of ethinyl estradiol per day for about 3 months after administration to the subject's vaginal tract. In another embodiment, the drug delivery system releases on an average about 0.001, 0.002, 0.003, 0.004, 0.005, 0.006, 0.007, 0.008, 0.009, 0.010, 0.012, and 0.013 mg of ethinyl estradiol per for day for about 21 days after administration to the subject's vaginal tract. In another embodiment, the drug delivery system releases on an average about 0.001, 0.002, 0.003, 0.004, 0.005, 0.006, 0.007, 0.008, 0.009, 0.010, 0.012, and 0.013 mg of ethinyl estradiol per for day for about 24 days after administration to the subject's vaginal tract. In another embodiment, the drug delivery system releases on an average about 0.001, 0.002, 0.003, 0.004, 0.005, 0.006, 0.007, 0.008, 0.009, 0.010, 0.012, and 0.013 mg of ethinyl estradiol per for day for about 26 days after administration to the subject's vaginal tract. In another embodiment, the drug delivery system releases on an average about 0.001, 0.002, 0.003, 0.004, 0.005, 0.006, 0.007, 0.008, 0.009, 0.010, 0.012, and 0.013 mg of ethinyl estradiol per for day for about 3 months after administration to the subject's vaginal tract. In a preferred embodiment, the system releases on an average about 0.010 mg of ethinyl estradiol per for day for about 21 days after administration to the subject's vaginal tract. In another preferred embodiment, the system releases on an average about 0.010 mg of ethinyl estradiol per for day for about 24 days after administration to the subject's vaginal tract. In another preferred embodiment, the system releases on an average about 0.010 mg of ethinyl estradiol per for day for about 26 days after administration to the subject's vaginal tract. In another preferred embodiment, the system releases on an average about 0.010 mg of ethinyl estradiol per for day for about 3 months after administration to the subject's vaginal tract.

In one embodiment, the drug delivery system includes from about 4 mg levonorgestrel to about 300 mg levonorgestrel. In one embodiment, the drug delivery system includes about 4, 10, 20, 30, 40, 50, 60, 70, 80, 90, 100, 110, 120, 130, 140, 150, 160, 170, 180, 190, 200, 210, 220, 230, 240, 250, 260, 270, 280, 290, or 300 mg of levonorgestrel. In one embodiment, the drug delivery system includes from about 0.1 mg to about 7.5 mg ethinyl estradiol. In another embodiment, the drug delivery system includes about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, or 7.5 mg of ethinyl estradiol. In a preferred embodiment, the drug delivery system includes about 49 mg of levonorgestrel and about 1.8 mg of ethinyl estradiol. In another preferred embodiment, the drug delivery system includes about 56 mg of levonorgestrel and about 2.1 mg of ethinyl estradiol. In another preferred embodiment, the drug delivery system includes about 60 mg of levonorgestrel and about 2.2 mg of ethinyl estradiol. In another preferred embodiment, the drug delivery system includes about 146 mg of levonorgestrel and about 5.4 mg of ethinyl estradiol. In one embodiment, the drug delivery system releases on an average from about 0.45 mg to about 0.55 mg of levonorgestrel per day for about 21 days. In one embodiment, the drug delivery system releases on an average from about 0.45 mg to about 0.55 mg of levonorgestrel per day for about 24 days. In one embodiment, the drug delivery system releases on an average from about 0.45 mg to about 0.55 mg of levonorgestrel per day for about 26 days. In one embodiment, the drug delivery system releases on an average from about 0.45 mg to about 0.55 mg of levonorgestrel per day for about 3 months. In another embodiment, the drug delivery system releases on an average about 0.45, 0.46, 0.47, 0.48, 0.49, 0.50, 0.51, 0.52, 0.53, 0.54 or 0.55 mg of levonorgestrel per for day for about 21 days after administration to the subject's vaginal tract. In another embodiment, the drug delivery system releases on an average about 0.45, 0.46, 0.47, 0.48, 0.49, 0.50, 0.51, 0.52, 0.53, 0.54 or 0.55 mg of levonorgestrel per for day for about 24 days after administration to the subject's vaginal tract. In another embodiment, the drug delivery system releases on an average about 0.45, 0.46, 0.47, 0.48, 0.49, 0.50, 0.51, 0.52, 0.53, 0.54 or 0.55 mg of levonorgestrel per for day for about 26 days after administration to the subject's vaginal tract. In another embodiment, the drug delivery system releases on an average about 0.45, 0.46, 0.47, 0.48, 0.49, 0.50, 0.51, 0.52, 0.53, 0.54 or 0.55 mg of levonorgestrel per for day for about 3 months after administration to the subject's vaginal tract. In a preferred embodiment, the system releases on an average about 0.50 mg of levonorgestrel per for day for about 21 days after administration to the subject's vaginal tract. In another preferred embodiment, the system releases on an average about 0.50 mg of levonorgestrel per for day for about 24 days after administration to the subject's vaginal tract. In a preferred embodiment, the system releases on an average about 0.50 mg of levonorgestrel per for day for about 26 days after administration to the subject's vaginal tract. In another preferred embodiment, the system releases on an average about 0.50 mg of levonorgestrel per for day for about 3 months after administration to the subject's vaginal tract. In one embodiment, the drug delivery system releases on an average from about 0.001 to about 0.013 mg of ethinyl estradiol per day for about 21 days after administration to the subject's vaginal tract. In one embodiment, the drug delivery system releases on an average from about 0.001 to about 0.013 mg of ethinyl estradiol per day for about 24 days after administration to the subject's vaginal tract. In one embodiment, the drug delivery system releases on an average from about 0.001 to about 0.013 mg of ethinyl estradiol per day for about 26 days after administration to the subject's vaginal tract. In one embodiment, the drug delivery system releases on an average from about 0.001 to about 0.013 mg of ethinyl estradiol per day for about 3 months after administration to the subject's vaginal tract. In another embodiment, the drug delivery system releases on an average about 0.001, 0.002, 0.003, 0.004, 0.005, 0.006, 0.007, 0.008, 0.009, 0.010, 0.012, and 0.013 mg of ethinyl estradiol per for day for about 21 days after administration to the subject's vaginal tract. In another embodiment, the drug delivery system releases on an average about 0.001, 0.002, 0.003, 0.004, 0.005, 0.006, 0.007, 0.008, 0.009, 0.010, 0.012, and 0.013 mg of ethinyl estradiol per for day for about 24 days after administration to the subject's vaginal tract. In another embodiment, the drug delivery system releases on an average about 0.001, 0.002, 0.003, 0.004, 0.005, 0.006, 0.007, 0.008, 0.009, 0.010, 0.012, and 0.013 mg of ethinyl estradiol per for day for about 26 days after administration to the subject's vaginal tract. In another embodiment, the drug delivery system releases on an average about 0.001, 0.002, 0.003, 0.004, 0.005, 0.006, 0.007, 0.008, 0.009, 0.010, 0.012, and 0.013 mg of ethinyl estradiol per for day for about 3 months after administration to the subject's vaginal tract. In a preferred embodiment, the system releases on an average about 0.010 mg of ethinyl estradiol per for day for about 21 days after administration to the subject's vaginal tract. In another preferred embodiment, the system releases on an average about 0.010 mg of ethinyl estradiol per for day for about 24 days after administration to the subject's vaginal tract. In another preferred embodiment, the system releases on an average about 0.010 mg of ethinyl estradiol per for day for about 26 days after administration to the subject's vaginal tract. In another preferred embodiment, the system releases on an average about 0.010 mg of ethinyl estradiol per for day for about 3 months after administration to the subject's vaginal tract.

In one embodiment, the steroidal progestogenic compound in the drug delivery system is drospirenone and the steroidal estrogenic compound in the drug delivery system is ethinyl estradiol. Such drug delivery system includes from about 150 mg to about 190 mg of drosprinone. In another embodiment, the drug delivery system includes about 150, 160, 170, 180, or 190 mg of drospirenone. This drug delivery system includes from about 0.1 mg to about 2.5 mg ethinyl estradiol. In another embodiment, the drug delivery system includes about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, or 2.5 mg of ethinyl estradiol. In a preferred embodiment, the drug delivery system includes about 300 mg of drospirenone and about 1.8 mg of ethinyl estradiol. In another preferred embodiment, the drug delivery system includes about 343 mg of drospirenone and about 2.1 mg of ethinyl estradiol. In another preferred embodiment, the drug delivery system includes about 371 mg of drospirenone and about 2.2 mg of ethinyl estradiol. In one embodiment, the drug delivery system releases on an average from about 0.1 mg to about 4 mg of drospirenone per day for about 21 days. In one embodiment, the drug delivery system releases on an average from about 0.1 mg to about 4 mg of drospirenone per day for about 24 days. In one embodiment, the drug delivery system releases on an average from about 0.1 mg to about 4 mg of drospirenone per day for about 26 days. In another embodiment, the drug delivery system releases on an average about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4 mg of drospirenone per for day for about 21 days after administration to the subject's vaginal tract. In another embodiment, the drug delivery system releases on an average about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4 mg of drospirenone per for day for about 24 days after administration to the subject's vaginal tract. In another embodiment, the drug delivery system releases on an average about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4 mg of drospirenone per for day for about 26 days after administration to the subject's vaginal tract. In a preferred embodiment, the system releases on an average about 3 mg of drospirenone per for day for about 21 days after administration to the subject's vaginal tract. In another preferred embodiment, the system releases on an average about 3 mg of drospirenone per for day for about 24 days after administration to the subject's vaginal tract. In another preferred embodiment, the system releases on an average about 1 mg of drospirenone per for day for about 26 days after administration to the subject's vaginal tract. In one embodiment, the drug delivery system releases on an average from about 0.001 to about 0.013 mg of ethinyl estradiol per day for about 21 days after administration to the subject's vaginal tract. In one embodiment, the drug delivery system releases on an average from about 0.001 to about 0.013 mg of ethinyl estradiol per day for about 24 days after administration to the subject's vaginal tract. In one embodiment, the drug delivery system releases on an average from about 0.001 to about 0.013 mg of ethinyl estradiol per day for about 26 days after administration to the subject's vaginal tract. In another embodiment, the drug delivery system releases on an average about 0.001, 0.002, 0.003, 0.004, 0.005, 0.006, 0.007, 0.008, 0.009, 0.010, 0.012, and 0.013 mg of ethinyl estradiol per for day for about 21 days after administration to the subject's vaginal tract. In another embodiment, the drug delivery system releases on an average about 0.001, 0.002, 0.003, 0.004, 0.005, 0.006, 0.007, 0.008, 0.009, 0.010, 0.012, and 0.013 mg of ethinyl estradiol per for day for about 24 days after administration to the subject's vaginal tract. In another embodiment, the drug delivery system releases on an average about 0.001, 0.002, 0.003, 0.004, 0.005, 0.006, 0.007, 0.008, 0.009, 0.010, 0.012, and 0.013 mg of ethinyl estradiol per for day for about 26 days after administration to the subject's vaginal tract. In a preferred embodiment, the system releases on an average about 0.010 mg of ethinyl estradiol per for day for about 21 days after administration to the subject's vaginal tract. In another preferred embodiment, the system releases on an average about 0.010 mg of ethinyl estradiol per for day for about 24 days after administration to the subject's vaginal tract. In another preferred embodiment, the system releases on an average about 0.010 mg of ethinyl estradiol per for day for about 26 days after administration to the subject's vaginal tract.

In one embodiment, the steroidal progestogenic compound in the drug delivery system is etonogestrel and the steroidal estrogenic compound in the drug delivery system is estradiol. Such drug delivery system includes from about 1 mg etonogestrel to about 75 mg etonogestrel. In one embodiment, the drug delivery system includes about 5, 10, 15, 20, 25, 30, 35, 40, 45, 50, 55, 60, 65, 70, or 75 mg of etonogestrel. In one embodiment, the drug delivery system includes from about 10 mg to about 105 mg of estradiol. In another embodiment, the drug delivery system includes about 10, 15, 20, 25, 30, 35, 40, 45, 50, 55, 60, 65, 70, 75, 80, 85, 90, 95, 100, or 105 mg of estradiol. In a preferred embodiment, the drug delivery system includes about 11.7 mg of etonogestrel and about 27 mg of estradiol. In another preferred embodiment, the drug delivery system includes about 35.1 mg of etonogestrel and about 81 mg of estradiol. In one embodiment, the drug delivery system releases on an average from about 0.1 mg to about 0.2 mg of etonogestrel per day for about 21 days. In one embodiment, the drug delivery system releases on an average from about 0.1 mg to about 0.2 mg of etonogestrel per day for about 24 days. In one embodiment, the drug delivery system releases on an average from about 0.1 mg to about 0.2 mg of etonogestrel per day for about 26 days. In one embodiment, the drug delivery system releases on an average from about 0.1 mg to about 0.2 mg of etonogestrel per day for about 3 months. In another embodiment, the drug delivery system releases on an average about 0.10, 0.11, 0.12, 0.13, 0.14, 0.15, 0.16, 0.17, 0.18, 0.19 or 0.20 mg of etonogestrel per for day for about 21 days after administration to the subject's vaginal tract. In another embodiment, the drug delivery system releases on an average about 0.10, 0.11, 0.12, 0.13, 0.14, 0.15, 0.16, 0.17, 0.18, 0.19 or 0.20 mg of etonogestrel per for day for about 24 days after administration to the subject's vaginal tract. In another embodiment, the drug delivery system releases on an average about 0.10, 0.11, 0.12, 0.13, 0.14, 0.15, 0.16, 0.17, 0.18, 0.19 or 0.20 mg of etonogestrel per for day for about 26 days after administration to the subject's vaginal tract. In another embodiment, the drug delivery system releases on an average about 0.10, 0.11, 0.12, 0.13, 0.14, 0.15, 0.16, 0.17, 0.18, 0.19 or 0.20 mg of etonogestrel per for day for about 3 months after administration to the subject's vaginal tract. In a preferred embodiment, the system releases on an average about 0.12 mg of etonogestrel per for day for about 21 days after administration to the subject's vaginal tract. In another preferred embodiment, the system releases on an average about 0.12 mg of etonogestrel per for day for about 24 days after administration to the subject's vaginal tract. In another preferred embodiment, the system releases on an average about 0.12 mg of etonogestrel per for day for about 26 days after administration to the subject's vaginal tract. In another preferred embodiment, the system releases on an average about 0.12 mg of etonogestrel per for day for about 3 months after administration to the subject's vaginal tract. In one embodiment, the drug delivery system releases on an average from about 0.05 to about 0.25 mg of estradiol per day for about 21 days after administration to the subject's vaginal tract. In one embodiment, the drug delivery system releases on an average from about 0.05 to about 0.25 mg of estradiol per day for about 24 days after administration to the subject's vaginal tract. In one embodiment, the drug delivery system releases on an average from about 0.05 to about 0.25 mg of estradiol per day for about 26 days after administration to the subject's vaginal tract. In one embodiment, the drug delivery system releases on an average from about 0.05 to about 0.25 mg of estradiol per day for about 3 months after administration to the subject's vaginal tract. In another embodiment, the drug delivery system releases on an average about 0.05, 0.1, 0.15, 0.2, or 0.25 mg of estradiol per for day for about 21 days after administration to the subject's vaginal tract. In another embodiment, the drug delivery system releases on an average about 0.05, 0.1, 0.15, 0.2, or 0.25 mg of estradiol per for day for about 24 days after administration to the subject's vaginal tract. In another embodiment, the drug delivery system releases on an average about 0.05, 0.1, 0.15, 0.2, or 0.25 mg of estradiol per for day for about 26 days after administration to the subject's vaginal tract. In another embodiment, the drug delivery system releases on an average about 0.05, 0.1, 0.15, 0.2, or 0.25 mg of estradiol per for day for about 3 months after administration to the subject's vaginal tract. In a preferred embodiment, the system releases on an average about 0.15 mg of estradiol per for day for about 21 days after administration to the subject's vaginal tract. In another preferred embodiment, the system releases on an average about 0.15 mg of estradiol per for day for about 24 days after administration to the subject's vaginal tract. In another preferred embodiment, the system releases on an average about 0.15 mg of estradiol per for day for about 26 days after administration to the subject's vaginal tract. In another preferred embodiment, the system releases on an average about 0.15 mg of estradiol per for day for about 3 months after administration to the subject's vaginal tract.

In one embodiment, the steroidal progestogenic compound in the drug delivery system is norethindrone acetate and the steroidal estrogenic compound in the drug delivery system is estradiol. Such drug delivery system includes from about 50 mg to about 750 mg of norethindrone acetate. In another embodiment, the drug delivery system includes about 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, or 750 mg of norethindrone acetate. In one embodiment, the drug delivery system includes from about 10 mg to about 105 mg of estradiol. In another embodiment, the drug delivery system includes about 10, 15, 20, 25, 30, 35, 40, 45, 50, 55, 60, 65, 70, 75, 80, 85, 90, 95, 100, or 105 mg of estradiol. In a preferred embodiment, the drug delivery system includes about 100 mg of norethindrone acetate and about 27 mg of estradiol. In another preferred embodiment, the drug delivery system includes about 300 mg of norethindrone acetate and about 81 mg of estradiol. In one embodiment, the drug delivery system releases on an average from about 0.1 mg to about 2 mg of norethindrone acetate per day for about 21 days. In one embodiment, the drug delivery system releases on an average from about 0.1 mg to about 2 mg of norethindrone acetate per day for about 24 days. In one embodiment, the drug delivery system releases on an average from about 0.1 mg to about 2 mg of norethindrone acetate per day for about 26 days. In one embodiment, the drug delivery system releases on an average from about 0.1 mg to about 2 mg of norethindrone acetate per day for about 3 months. In another embodiment, the drug delivery system releases on an average about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, or 2 mg of norethindrone acetate per for day for about 21 days after administration to the subject's vaginal tract. In another embodiment, the drug delivery system releases on an average about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, or 2 mg of norethindrone acetate per for day for about 24 days after administration to the subject's vaginal tract. In another embodiment, the drug delivery system releases on an average about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, or 2 mg of norethindrone acetate per for day for about 26 days after administration to the subject's vaginal tract. In another embodiment, the drug delivery system releases on an average about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, or 2 mg of norethindrone acetate per for day for about 3 months after administration to the subject's vaginal tract. In a preferred embodiment, the system releases on an average about 1 mg of norethindrone acetate per for day for about 21 days after administration to the subject's vaginal tract. In another preferred embodiment, the system releases on an average about 1 mg of norethindrone acetate per for day for about 24 days after administration to the subject's vaginal tract. In another preferred embodiment, the system releases on an average about 1 mg of norethindrone acetate per for day for about 26 days after administration to the subject's vaginal tract. In another preferred embodiment, the system releases on an average about 1 mg of norethindrone acetate per for day for about 3 months after administration to the subject's vaginal tract. In one embodiment, the drug delivery system releases on an average from about 0.05 to about 0.25 mg of estradiol per day for about 21 days after administration to the subject's vaginal tract. In one embodiment, the drug delivery system releases on an average from about 0.05 to about 0.25 mg of estradiol per day for about 24 days after administration to the subject's vaginal tract. In one embodiment, the drug delivery system releases on an average from about 0.05 to about 0.25 mg of estradiol per day for about 26 days after administration to the subject's vaginal tract. In one embodiment, the drug delivery system releases on an average from about 0.05 to about 0.25 mg of estradiol per day for about 3 months after administration to the subject's vaginal tract. In another embodiment, the drug delivery system releases on an average about 0.05, 0.1, 0.15, 0.2, or 0.25 mg of estradiol per for day for about 21 days after administration to the subject's vaginal tract. In another embodiment, the drug delivery system releases on an average about 0.05, 0.1, 0.15, 0.2, or 0.25 mg of estradiol per for day for about 24 days after administration to the subject's vaginal tract. In another embodiment, the drug delivery system releases on an average about 0.05, 0.1, 0.15, 0.2, or 0.25 mg of estradiol per for day for about 26 days after administration to the subject's vaginal tract. In another embodiment, the drug delivery system releases on an average about 0.05, 0.1, 0.15, 0.2, or 0.25 mg of estradiol per for day for about 3 months after administration to the subject's vaginal tract. In a preferred embodiment, the system releases on an average about 0.15 mg of estradiol per for day for about 21 days after administration to the subject's vaginal tract. In another preferred embodiment, the system releases on an average about 0.15 mg of estradiol per for day for about 24 days after administration to the subject's vaginal tract. In another preferred embodiment, the system releases on an average about 0.15 mg of estradiol per for day for about 26 days after administration to the subject's vaginal tract. In another preferred embodiment, the system releases on an average about 0.15 mg of estradiol per for day for about 3 months after administration to the subject's vaginal tract.

In one embodiment, the drug delivery system includes from about 4 mg levonorgestrel to about 300 mg levonorgestrel. In one embodiment, the drug delivery system includes about 4, 10, 20, 30, 40, 50, 60, 70, 80, 90, 100, 110, 120, 130, 140, 150, 160, 170, 180, 190, 200, 210, 220, 230, 240, 250, 260, 270, 280, 290, or 300 mg of levonorgestrel. In one embodiment, the drug delivery system includes from about 10 mg to about 105 mg of estradiol. In another embodiment, the drug delivery system includes about 10, 15, 20, 25, 30, 35, 40, 45, 50, 55, 60, 65, 70, 75, 80, 85, 90, 95, 100, or 105 mg of estradiol. In a preferred embodiment, the drug delivery system includes about 49 mg of levonorgestrel and about 27 mg of estradiol. In another preferred embodiment, the drug delivery system includes about 56 mg of levonorgestrel and about 31 mg of estradiol. In another preferred embodiment, the drug delivery system includes about 60 mg of levonorgestrel and about 33 mg of estradiol. In another preferred embodiment, the drug delivery system includes about 146 mg of levonorgestrel and about 81 mg of estradiol. In one embodiment, the drug delivery system releases on an average from about 0.45 mg to about 0.55 mg of levonorgestrel per day for about 21 days. In one embodiment, the drug delivery system releases on an average from about 0.45 mg to about 0.55 mg of levonorgestrel per day for about 24 days. In one embodiment, the drug delivery system releases on an average from about 0.45 mg to about 0.55 mg of levonorgestrel per day for about 26 days. In one embodiment, the drug delivery system releases on an average from about 0.45 mg to about 0.55 mg of levonorgestrel per day for about 3 months. In another embodiment, the drug delivery system releases on an average about 0.45, 0.46, 0.47, 0.48, 0.49, 0.50, 0.51, 0.52, 0.53, 0.54 or 0.55 mg of levonorgestrel per for day for about 21 days after administration to the subject's vaginal tract. In another embodiment, the drug delivery system releases on an average about 0.45, 0.46, 0.47, 0.48, 0.49, 0.50, 0.51, 0.52, 0.53, 0.54 or 0.55 mg of levonorgestrel per for day for about 24 days after administration to the subject's vaginal tract. In another embodiment, the drug delivery system releases on an average about 0.45, 0.46, 0.47, 0.48, 0.49, 0.50, 0.51, 0.52, 0.53, 0.54 or 0.55 mg of levonorgestrel per for day for about 26 days after administration to the subject's vaginal tract. In another embodiment, the drug delivery system releases on an average about 0.45, 0.46, 0.47, 0.48, 0.49, 0.50, 0.51, 0.52, 0.53, 0.54 or 0.55 mg of levonorgestrel per for day for about 3 months after administration to the subject's vaginal tract. In a preferred embodiment, the system releases on an average about 0.50 mg of levonorgestrel per for day for about 21 days after administration to the subject's vaginal tract. In another preferred embodiment, the system releases on an average about 0.50 mg of levonorgestrel per for day for about 24 days after administration to the subject's vaginal tract. In another preferred embodiment, the system releases on an average about 0.50 mg of levonorgestrel per for day for about 26 days after administration to the subject's vaginal tract. In another preferred embodiment, the system releases on an average about 0.50 mg of levonorgestrel per for day for about 3 months after administration to the subject's vaginal tract. In one embodiment, the drug delivery system releases on an average from about 0.05 to about 0.25 mg of estradiol per day for about 21 days after administration to the subject's vaginal tract. In one embodiment, the drug delivery system releases on an average from about 0.05 to about 0.25 mg of estradiol per day for about 24 days after administration to the subject's vaginal tract. In one embodiment, the drug delivery system releases on an average from about 0.05 to about 0.25 mg of estradiol per day for about 26 days after administration to the subject's vaginal tract. In one embodiment, the drug delivery system releases on an average from about 0.05 to about 0.25 mg of estradiol per day for about 3 months after administration to the subject's vaginal tract. In another embodiment, the drug delivery system releases on an average about 0.05, 0.1, 0.15, 0.2, or 0.25 mg of estradiol per for day for about 21 days after administration to the subject's vaginal tract. In another embodiment, the drug delivery system releases on an average about 0.05, 0.1, 0.15, 0.2, or 0.25 mg of estradiol per for day for about 24 days after administration to the subject's vaginal tract. In another embodiment, the drug delivery system releases on an average about 0.05, 0.1, 0.15, 0.2, or 0.25 mg of estradiol per for day for about 26 days after administration to the subject's vaginal tract. In another embodiment, the drug delivery system releases on an average about 0.05, 0.1, 0.15, 0.2, or 0.25 mg of estradiol per for day for about 3 months after administration to the subject's vaginal tract. In a preferred embodiment, the system releases on an average about 0.15 mg of estradiol per for day for about 21 days after administration to the subject's vaginal tract. In another preferred embodiment, the system releases on an average about 0.15 mg of estradiol per for day for about 24 days after administration to the subject's vaginal tract. In another preferred embodiment, the system releases on an average about 0.15 mg of estradiol per for day for about 26 days after administration to the subject's vaginal tract. In another preferred embodiment, the system releases on an average about 0.15 mg of estradiol per for day for about 3 months after administration to the subject's vaginal tract.

In one embodiment, the steroidal progestogenic compound in the drug delivery system is drospirenone and the steroidal estrogenic compound in the drug delivery system is estradiol. Such drug delivery system includes from about 150 mg to about 190 mg of drosprinone. In another embodiment, the drug delivery system includes about 150, 160, 170, 180, or 190 mg of drospirenone. In one embodiment, the drug delivery system includes from about 10 mg to about 35 mg of estradiol. In another embodiment, the drug delivery system includes about 10, 15, 20, 25, 30, or 35 mg of estradiol. In a preferred embodiment, the drug delivery system includes about 300 mg of drospirenone and about 27 mg of estradiol. In another preferred embodiment, the drug delivery system includes about 343 mg of drospirenone and about 31 mg of estradiol. In another preferred embodiment, the drug delivery system includes about 371 mg of drospirenone and about 33 mg of estradiol. In one embodiment, the drug delivery system releases on an average from about 0.1 mg to about 4 mg of drospirenone per day for about 21 days. In one embodiment, the drug delivery system releases on an average from about 0.1 mg to about 4 mg of drospirenone per day for about 24 days. In one embodiment, the drug delivery system releases on an average from about 0.1 mg to about 4 mg of drospirenone per day for about 26 days. In another embodiment, the drug delivery system releases on an average about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4 mg of drospirenone per for day for about 21 days after administration to the subject's vaginal tract. In another embodiment, the drug delivery system releases on an average about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4 mg of drospirenone per for day for about 24 days after administration to the subject's vaginal tract. In another embodiment, the drug delivery system releases on an average about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4 mg of drospirenone per for day for about 26 days after administration to the subject's vaginal tract. In a preferred embodiment, the system releases on an average about 3 mg of drospirenone per for day for about 21 days after administration to the subject's vaginal tract. In another preferred embodiment, the system releases on an average about 3 mg of drospirenone per for day for about 24 days after administration to the subject's vaginal tract. In another preferred embodiment, the system releases on an average about 1 mg of drospirenone per for day for about 26 days after administration to the subject's vaginal tract. In one embodiment, the drug delivery system releases on an average from about 0.05 to about 0.25 mg of estradiol per day for about 21 days after administration to the subject's vaginal tract. In one embodiment, the drug delivery system releases on an average from about 0.05 to about 0.25 mg of estradiol per day for about 24 days after administration to the subject's vaginal tract. In one embodiment, the drug delivery system releases on an average from about 0.05 to about 0.25 mg of estradiol per day for about 26 days after administration to the subject's vaginal tract. In another embodiment, the drug delivery system releases on an average about 0.05, 0.1, 0.15, 0.2, or 0.25 mg of estradiol per for day for about 21 days after administration to the subject's vaginal tract. In another embodiment, the drug delivery system releases on an average about 0.05, 0.1, 0.15, 0.2, or 0.25 mg of estradiol per for day for about 24 days after administration to the subject's vaginal tract. In another embodiment, the drug delivery system releases on an average about 0.05, 0.1, 0.15, 0.2, or 0.25 mg of estradiol per for day for about 26 days after administration to the subject's vaginal tract. In a preferred embodiment, the system releases on an average about 0.15 mg of estradiol per for day for about 21 days after administration to the subject's vaginal tract. In another preferred embodiment, the system releases on an average about 0.15 mg of estradiol per for day for about 24 days after administration to the subject's vaginal tract. In another preferred embodiment, the system releases on an average about 0.15 mg of estradiol per for day for about 26 days after administration to the subject's vaginal tract.

The drug delivery system as disclosed herein can have different shapes or forms, such as, an implant, a sphere, a cylinder, an intrauterine system, a helical coil, a toroid, a spring, or a ring-shaped vaginal drug delivery system. When the drug delivery system is for intra-vaginal use it may be used for female medical indications, such as, for contraception.

In one embodiment the drug delivery system, e.g., the vaginal ring has multiple sections and the active agents of the invention are present in different sections present within the ring. For instance, in an embodiment, the steroidal estrogenic compound and a steroidal progestogenic compound are enclosed in temporary or permanent separate sections of the core. In another embodiment, the steroidal estrogenic compound and a steroidal progestogenic compound are enclosed in the same section of the core. In another embodiment, the steroidal estrogenic compound and a steroidal progestogenic compound are enclosed in same and/or temporary or permanent separate compartments within the core.

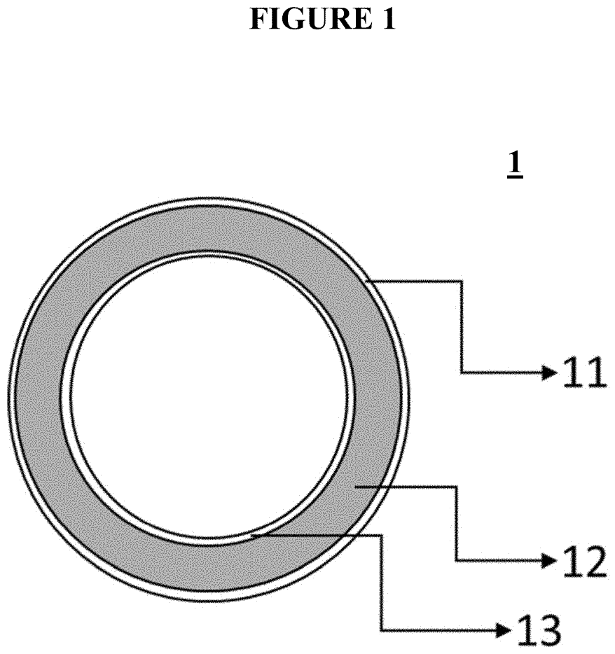

The drug delivery system, e.g., as shown in FIG. 1, can be in the form of a vaginal ring. The vaginal rings of this invention have the advantage that the active agents are released therefrom for a longer period of time (or total time period) regularly and uniformly within the limits of the dosage required for the desired biological effect, e.g. for the inhibition of ovulation. Moreover, these vaginal rings have the advantage that the amount of active agent which must be incorporated into a single such contraceptive can be much lower than in the case of the conventional vaginal rings.

In one embodiment, the vaginal ring can be an annular ring that includes a core (12) and an outer skin (also called membrane) (11 and 13) as shown by the exemplary drug delivery system (1) in FIG. 1. The active agents described in the invention are included in the core (12) of the ring and are in a form whereby they can be released from the core (12) of the ring to the subject's vaginal tract upon administration of the ring to the subject's vaginal tract. For instance, when the ring as shown in FIG. 1 is administered to the subject, the ring releases or delivers the active ingredients to the subject's vaginal tract. The skin (11 and 13) can be made such that it controls the release rate of the active agents included within the core (12) of the ring. In one embodiment, the active agents included in the vaginal ring are released from the core (12) of the drug delivery system (1) such that they are absorbed by the vaginal mucosa of the subject.

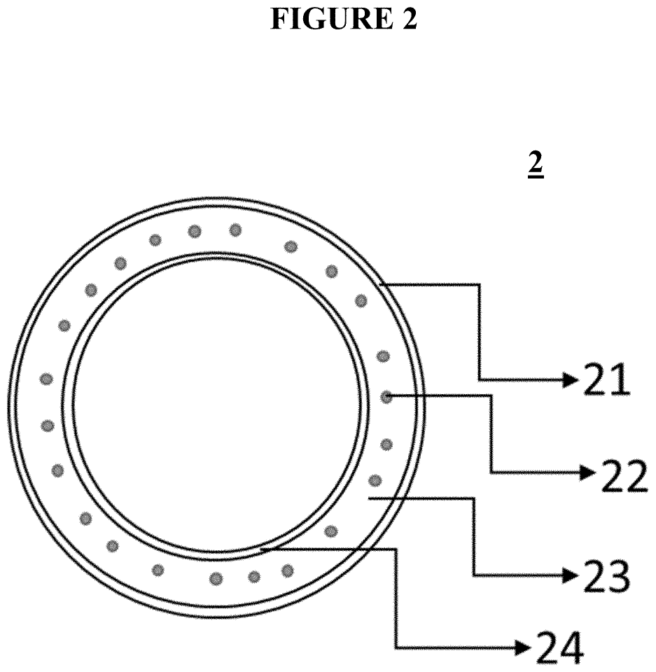

In one embodiment, the drug delivery system is in the form of a ring, as shown by the exemplary drug delivery system (2) in FIG. 2, where the core (23) includes the active agents in the form of granules, beads, spheres, crystals or the like (22). The granules of the active agent are such that they are capable of delivering the active agent to a subject upon administration of the ring to the subject's vaginal tract. The core (23) of the ring may be optionally covered by a membrane (21 and 24). The membrane controls the release rate of the active agents included within the core (23) of the ring in exemplary drug delivery system (2).

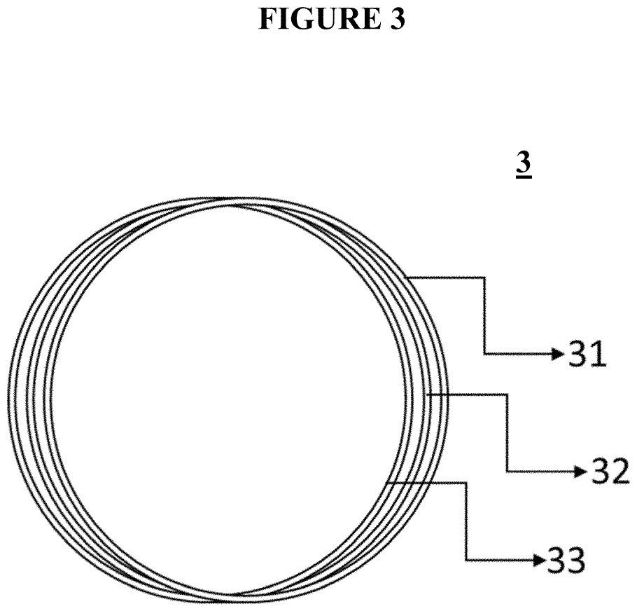

In another embodiment, the drug delivery system is in the form of multiple annular rings that may be attached to one another, concatenated, or connected to one another, as shown by the exemplary drug delivery system (3) in FIG. 3. One or more rings (31, 32, and 33) may include a separate active agent dispersed within the one or more rings (31, 32 and 33, respectively). One or more rings (31, 32, and 33) may optionally include a membrane over the surface of the central core portion of the one or more rings (31, 32, and 33) where the membrane controls the rate of release of the active agent included within the one or more rings (31, 32, and 33).

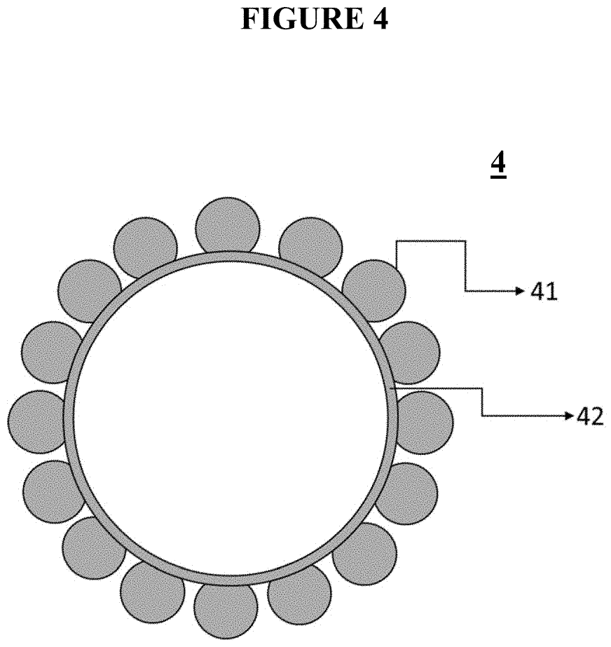

In another embodiment, the vaginal ring includes a retaining annular ring (42) in the center and a plurality of pockets or compartments (41) attached to the outer portion the retaining annular ring (42), as shown by the exemplary drug delivery system (4) in FIG. 4. Each pocket or compartment (41) as well as the retaining annular ring (42) may include an active ingredient and is such that the active ingredient may be delivered to the subject upon administration of the vaginal ring to the subject's vaginal tract.

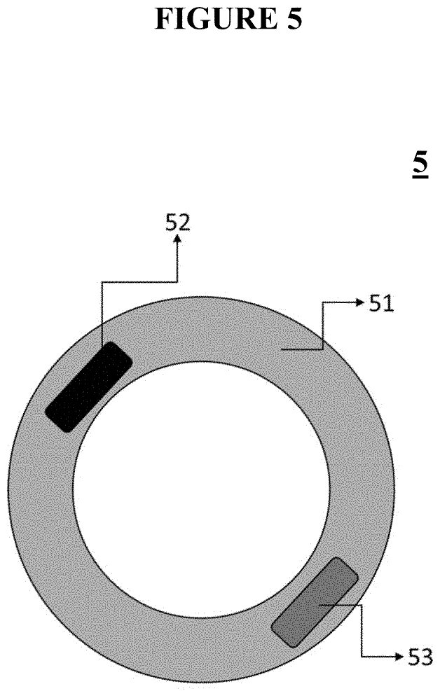

In another embodiment, the vaginal ring includes a retaining annular ring (51) and a plurality of pockets or compartments (52, 53) within the retaining annular ring (51), as shown by the exemplary drug delivery system (5) in FIG. 5. Each pocket or compartment (52, 53) may include an active ingredient and is such that the active ingredient may be delivered to the subject upon administration of the vaginal ring to the subject's vaginal tract.

Additionally, in some instances, the vaginal rings of the invention may be in the shape or form of the vaginal rings disclosed in U.S. Pat. Nos. 3,995,633; 3,995,634; 4,292,965; 4,596,576; and 5,989,581, all of which are hereby incorporated by reference in their entirety.

The drug delivery system according to the invention can be manufactured in any suitable manner know in the art, e.g., like the ones disclosed in e.g. U.S. Pat. Nos. 3,995,633; 3,99,634; 4,292,965; 4,596,576; and 5,989,581, all of which are hereby incorporated by reference in their entirety. A preferred method of manufacture of the vaginal rings includes co-extrusion of a drug-loaded core and the non-medicated outer layer. The fibers thus obtained are cut into pieces of the required length and each piece is assembled to a ring shaped device in any suitable manner. The rings are then packed for example in a suitable sachet, optionally, after being sterilized or disinfected.

The vaginal rings of the invention provide for the reliable and predictable release of the steroid compounds and active agents of the invention. In one embodiment, the vaginal ring includes a thermoplastic polymer core or an elastomer core, optionally, containing at least a steroidal progestogenic compound and a steroidal estrogenic compound.

The polymer core allows direct release of both the progestogenic compound and the estrogenic compound in physiologically required amounts. In one embodiment, the progestogenic compound is dissolved in the core polymer at a relatively low degree of supersaturation, preferably between about 1 to about 6 times of the amount by weight necessary for obtaining the saturation concentration of said progestogenic steroid in said core polymer at 25° C. In another embodiment, the estrogenic compound is dissolved in the core polymer at a concentration that is lower than that of the said progestogenic compound. The vaginal ring, in one embodiment, has a thermoplastic skin or membrane (outer layer) that is permeable to the progestogenic and estrogenic compounds.

The thermoplastic polymer that can be used in practicing the invention may in principle be any thermoplastic polymer or elastomer material suitable for pharmaceutical use, such as ethylene-vinyl acetate copolymers, low-density polyethylene, styrene-butadiene-styrene copolymers, thermoplastic polyurethanes or a combination thereof. The ethylene-vinyl acetate copolymer (poly-EVA) is highly preferred due to its excellent mechanical and physical properties (e.g., solubility of the steroids in the material). The poly-EVA material may preferably be used for both the core as well as the membrane/skin and can be any commercially available ethylene-vinyl acetate copolymer, such as the products available under the trade names: Ativa®, VitalDose®, Elvax®, Evatane®, Lupolen®, Movriton®, Ultrathene® and Vestypar®. The membrane/skin of the vaginal ring can, e.g., also be made of olefins, vinyl polymers, rubber polymers, silicon polymers, microporous polymers, diffusion polymers, polyethylene, ethylene-vinyl acetate copolymers, polyether-ester polymers, cellulose, or combinations thereof.

The membrane or skin, as disclosed in the invention, has excellent solubility and steroid diffusion properties, allowing for release of the steroidal estrogenic compound, the steroidal progestogenic compound, or a combination thereof. In a preferred embodiment, the skin/membrane allows for the combined release of the steroidal estrogenic compound and the steroidal progestogenic compound in the proper ratio at moderate concentrations of the steroids in the vaginal ring during a period of time (or total time period).

The drug delivery system according to the invention can have any size, shape, and form as required by, e.g., its function, its use, and anatomy of a subject who is a recipient of the drug delivery system. In a preferred embodiment, the drug delivery system has a size, shape and form that are adapted for easy placement in, retention in and easy removal from a subject's vaginal tract.

In another preferred embodiment, the vaginal ring according to the invention can be manufactured in any size as required. In practice, however, the ring has an outer diameter of between about 50 and about 60 mm and more preferably between about 52 and about 56 mm; the cross sectional diameter of the vaginal ring is preferably between about 2.5 and 5 mm.

The vaginal rings preferably have a circular cross section. The cross section, however, can also be of the shape of a square, rectangle, oval, figure-eight, triangle, or a combination thereof. In one embodiment, the dimensions of the vaginal ring are selected so that it maintains contact points with the vaginal mucosa or the fluid present in the vaginal tract of the subject upon administration the subject's vaginal tract.

The size of the vaginal ring is dependent on the species for utilization of the ring, e.g., for smaller mammals, such as dogs, the ring size will be smaller than in the case of larger mammals such as horses and cows. In vaginal rings for use in women, the outer diameter is between about 50 to 60 mm, and in the case of Rhesus monkeys, for example, between about 20 to 30 mm. The diameter of such ring cross sections is generally between about 2.5 to about 5 mm.

Another aspect of the invention is related to a method of contraception. This method includes a step of retainably positioning the drug delivery system, e.g., the vaginal ring within a subject's vaginal tract; retaining the system within the subject's vaginal tract for a period of time; and removing the system from the subject after the period of time (or total period of time). In one embodiment, the period of time (or total period of time) is about 21 days. In one embodiment, the period of time (or total period of time) is about 24 days. In one embodiment, the period of time (or total period of time) is about 26 days. In one embodiment, the period of time (or total period of time) is about 3 months. In yet another embodiment, the method can include intermittent removal and reinsertion of the drug delivery system at prescribed intervals for prescribed periods of time.

Vaginal rings of the invention are, in one instance, meant to be introduced into the vagina tract of a subject in a simple manner without medical assistance. The vaginal ring fits between the rear wall of the vagina and the upper edge of the pubic bone. The vaginal ring of the invention is primarily useful for the inhibition of fertility, which is contraceptive purposes, but in some instances may also be used to treat and medicate other conditions. The vaginal rings are designed so that they can be retained in the vagina for a period about 21 days to about one year. In one embodiment, the ring is inserted in the vagina for 3 weeks, removed for one week and reinserted on a schedule of three weeks in, one week out, for a period of time between 1 month to 1 year, 2 months to 6 months, or, more preferably for 3 months.

The vaginal rings contain medication or active agents, for example, effective amount of contraceptive steroids, such as steroidal estrogenic compounds or steroidal progestogenic compounds, which diffuse through the vaginal rings and are absorbed by the surrounding body fluid through the vaginal mucosa or by via the vaginal mucosa. The vaginal ring exerts its medicative or contraception effect as long as the vaginal ring is retained within the body or the supply of the medication in the vaginal ring or the subject's body is sufficient for its indicated purpose. The concept of using vaginal rings as a means of administering effective contraceptive steroids by absorption through the vaginal mucosa for contraceptive purposes was tested by Mishell and associates in 1970, Mishell, D. R. Jr., et al. Am. J. Obstet. Gynecol. 107:100, 1970, which is hereby incorporated by reference in its entirety.

Yet another aspect of the invention is related to a method of making the drug delivery system. This method includes a step of producing a core comprising the combination of a steroidal estrogenic compound and a steroidal progestogenic compound and a subsequent step of covering a portion or all of the (the entire) core with a membrane or a skin to form the drug delivery system. In one embodiment, the core of the drug delivery system is coextruded with the membrane or the skin to form the drug delivery system.

The vaginal ring according to the invention can be manufactured in any suitable manner, e.g., as disclosed in U.S. Pat. Nos. 5,989,581; 4,012,496; and 4,292,965. A preferred method of manufacture comprises co-extrusion of the drug-loaded core and the non-medicated outer layer. The fibers thus obtained are cut into pieces of the required length and each piece is assembled to a ring shaped device in any suitable manner. The rings are then packed for example in a suitable sachet, optionally after being sterilized or disinfected.

The vaginal ring according to the invention is primarily designed for contraceptive use, but as said above may also be used under certain conditions.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Variations of certain described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

In closing, it is to be understood that the embodiments of the invention disclosed herein are illustrative of the principles of the invention. Other modifications that may be employed are within the scope of the invention. Thus, by way of example, but not of limitation, alternative configurations of the invention may be utilized in accordance with the teachings herein. Accordingly, the invention is not limited to that precisely as shown and described.

What is claimed is:

1. A drug delivery system for use in contraception by administering the system to a subject's vaginal tract, said system comprising:
a) a core comprising a steroidal estrogenic compound and a steroidal progestogenic compound;
b) a membrane covering a portion or all of the core;
wherein, when the system is in the subject's vaginal tract, the system delivers a therapeutically effective dose of the steroidal estrogenic compound and a therapeutically effective dose of the steroidal progestogenic compound to the subject for a period of time to produce a contraceptive effect in the subject, and the system delivers less than 0.013 mg per day of the steroidal estrogenic compound to the subject's vaginal tract.

2. The drug delivery system according to claim 1, wherein the period of time is about 21 days, about 24 days, about 26 days, or about 3 months after administration to the subject's vaginal tract.

3. The drug delivery system according to claim 1, wherein the system delivers the therapeutically effective dose of the steroidal estrogenic compound and the therapeutically effective dose of steroidal progestogenic compound to the subject at a controlled rate for the period of time, and the period of time is about 21 days, about 24 days, about 26 days, or about 3 months after administration to the subject's vaginal tract.

4. The drug delivery system according to claim 1, wherein the steroidal estrogenic compound is ethinyl estradiol or estradiol.

5. The drug delivery system according to claim 4, wherein the system delivers about 0.010 mg per day of ethinyl estradiol to the subject for about 21 days, about 24 days, about 26 days, or about 3 months after administration to the subject's vaginal tract.

6. The drug delivery system according to claim 1, wherein the steroidal progestogenic compound is selected from a group consisting of drospirenone, norethindrone, norethindrone acetate, levonorgestrel, etonogestrel, and norgestimate.

7. The drug delivery system according to claim 1, wherein the steroidal progestogenic compound is etonogestrel and the steroidal estrogenic compound is ethinyl estradiol.

8. The drug delivery system according to claim 7, wherein the amount of etonogestrel in the drug delivery system is about 11.7 mg and the amount of ethinyl estradiol in the drug delivery system is about 1.8 mg, or the amount of etonogestrel in the drug delivery system is about 35.1 mg and the amount of ethinyl estradiol in the drug delivery system is about 5.4 mg.

9. The drug delivery system according to claim 7, wherein the system releases on an average about 0.12 mg of etonogestrel per day for about 21 days, about 24 days, about 26 days, or about 3 months after administration of the system to the subject's vaginal tract.

10. The drug delivery system according to claim 7, wherein the system releases on an average about 0.010 mg of ethinyl estradiol per day for about 21 days, about 24 days, about 26 days, or about 3 months after administration of the system to the subject's vaginal tract.

11. The drug delivery system according to claim 1, wherein the steroidal progestogenic compound is norethindrone acetate and the steroidal estrogenic compound is ethinyl estradiol.

12. The drug delivery system according to claim 11, wherein the amount of norethindrone acetate in the drug delivery system is about 100 mg and the amount of ethinyl estradiol in the drug delivery system is about 1.8 mg, or the amount of norethindrone acetate in the drug delivery system is about 300 mg and the amount of ethinyl estradiol in the drug delivery system is about 5.4 mg.

13. The drug delivery system according to claim 11, wherein the system releases on an average about 1 mg of norethindrone acetate per day for about 21 days, about 24 days, about 26 days, or about 3 months after administration of the drug delivery system to the subject's vaginal tract.

14. The drug delivery system according to claim 11, wherein the system releases on an average about 0.010 mg of ethinyl estradiol per day for about 21 days, about 24 days, about 26 days, or about 3 months after administration of the drug delivery system to the subject's vaginal tract.

15. The drug delivery system according to claim 1, wherein the steroidal progestogenic compound is etonogestrel and the steroidal estrogenic compound is estradiol.

16. The drug delivery system according to claim 15, wherein the amount of etonogestrel in the drug delivery system is about 11.7 mg and the amount of estradiol in the drug delivery system is about 27 mg, or the amount of etonogestrel in the drug delivery system is about 35.1 mg and the amount of estradiol in the drug delivery system is about 81 mg.

17. The drug delivery system according to claim 15, wherein the drug delivery system releases on an average about 0.12 mg of etonogestrel per day for about 21 days, about 24 days, about 26 days, or about 3 months after administration of the drug delivery system to the subject's vaginal tract.

18. The drug delivery system according to claim 15, wherein the drug delivery system releases on an average about 0.15 mg of estradiol per day for about 21 days, about 24 days, about 26 days, or about 3 months after administration of the drug delivery system to the subject's vaginal tract.

19. The drug delivery system according to claim 1, wherein the steroidal progestogenic compound is norethindrone acetate and the steroidal estrogenic compound is estradiol.

20. The drug delivery system according to claim 19, wherein the amount of norethindrone acetate in the drug delivery system is about 100 mg and the amount of estradiol in the drug delivery system is about 27 mg, or the amount of norethindrone acetate in the drug delivery system is about 300 mg and the amount of estradiol in the drug delivery system is about 81 mg.

21. The drug delivery system according to claim 19, wherein the drug delivery system releases on an average about 1 mg of norethindrone acetate per day for about 21 days, about 24 days, about 26 days, or about 3 months after administration of the drug delivery system to the subject's vaginal tract.

22. The drug delivery system according to claim 19, wherein the drug delivery system releases on an average about 0.15 mg of estradiol per day for about 21 days, about 24 days, about 26 days, or about 3 months after administration of the drug delivery system to the subject's vaginal tract.

23. The drug delivery system according to claim 1, wherein the steroidal progestogenic compound is levonorgestrel or drospirenone and the steroidal estrogenic compound is ethinyl estradiol.

24. The drug delivery system according to claim 23, wherein the amount of levonorgestrel in the drug delivery system is about 49 mg and the amount of ethinyl estradiol in the drug delivery system is about 1.8 mg, or the amount of levonorgestrel in the drug delivery system is about 56 mg and the amount of ethinyl estradiol in the drug delivery system is about 2.1 mg, or the amount of levonorgestrel in the drug delivery system is about 60 mg and the amount of ethinyl estradiol in the drug delivery system is about 2.2 mg, or the amount of levonorgestrel in the drug delivery system is about 146 mg and the amount of ethinyl estradiol in the drug delivery system is about 5.4 mg,

25. The drug delivery system according to claim 23, wherein the system releases on an average about 0.50 mg of levonorgestrel per day for about 21 days, about 24 days, about 26 days, or about 3 months after administration of the system to the subject's vaginal tract.

26. The drug delivery system according to claim 23, wherein the system releases on an average about 0.010 mg of ethinyl estradiol per day for about 21 days, about 24 days, about 26 days, or about 3 months after administration of the system to the subject's vaginal tract.

27. The drug delivery system according to claim 23, wherein the amount of drospirenone in the drug delivery system is about 300 mg and the amount of ethinyl estradiol in the drug delivery system is about 1.8 mg, or the amount of drospirenone in the drug delivery system is about 343 mg and the amount of ethinyl estradiol in the drug delivery system is about 2.1 mg, or the amount of drospirenone in the drug delivery system is about 371 mg and the amount of ethinyl estradiol in the drug delivery system is about 2.2 mg.

28. The drug delivery system according to claim 23, wherein the system releases on an average about 3 mg of drospirenone per day for about 21 days or about 24 days after administration of the system to the subject's vaginal tract, or an average about 1 mg of drospirenone per day for about 26 days after administration of the system to the subject's vaginal tract.

29. The drug delivery system according to claim 28, wherein the system releases on an average about 0.010 mg of ethinyl estradiol per day for about 21 days, about 24 days, or about 26 days after administration of the system to the subject's vaginal tract.

30. The drug delivery system according to claim 1, wherein the steroidal progestogenic compound is levonorgestrel or drospirenone and the steroidal estrogenic compound is estradiol.

31. The drug delivery system according to claim 30, wherein the amount of levonorgestrel in the drug delivery system is about 49 mg and the amount of estradiol in the drug delivery system is about 27 mg, or the amount of levonorgestrel in the drug delivery system is about 56 mg and the amount of estradiol in the drug delivery system is about 31 mg, or the amount of levonorgestrel in the drug delivery system is about 60 mg and the amount of estradiol in the drug delivery system is about 33 mg, or the amount of levonorgestrel in the drug delivery system is about 146 mg and the amount of estradiol in the drug delivery system is about 81 mg.

32. The drug delivery system according to claim 30, wherein the system releases on an average about 0.5 mg of levonorgestrel per day for about 21 days, about 24 days, about 26 days, or about 3 months after administration of the system to the subject's vaginal tract.

33. The drug delivery system according to claim 30, wherein the system releases on an average about 0.15 mg of estradiol per day for about 21 days, about 24 days, about 26 days, or about 3 months after administration of the system to the subject's vaginal tract.

34. The drug delivery system according to claim 30, wherein the amount of drospirenone in the drug delivery system is about 300 mg and the amount of estradiol in the drug delivery system is about 27 mg, or the amount of drospirenone in the drug delivery system is about 343 mg and the amount of estradiol in the drug delivery system is about 31 mg, or the amount of drospirenone in the drug delivery system is about 371 mg and the amount of estradiol in the drug delivery system is about 33 mg.

35. The drug delivery system according to claim 30, wherein the system releases on an average about 3 mg of drospirenone per day for about 21 days or about 24 days after administration of the system to the subject's vaginal tract, or an average about 1 mg of drospirenone per day for about 26 days after administration of the system to the subject's vaginal tract.

36. The drug delivery system according to claim 35, wherein the system releases on an average about 0.15 mg of estradiol per day for about 21 days, about 24 days, or about 26 days after administration of the system to the subject's vaginal tract.

37. The drug delivery system according to claim 1, wherein the core is made of a thermoplastic polymer or an elastomer.

38. The drug delivery system according to claim 1, wherein the core is made of a material selected from the group consisting of low-density polyethylene, ethylene-vinyl acetate copolymers, thermoplastic polyurethane, styrene-butadiene-styrene copolymers, and a combination thereof.

39. The drug delivery system according to claim 1, wherein the membrane is made of a material selected from the group consisting of olefins, vinyl polymers, rubber polymers, silicon polymers, microporous polymers, diffusion polymers, polyethylene, ethylene-vinyl acetate copolymers, thermoplastic polyurethanes, polyether-ester polymers, cellulose, and a combination thereof.

40. The drug delivery system according to claim 1, wherein the delivery system is adapted for administration in the subject's vaginal tract.

41. The drug delivery system according to claim 40, wherein the delivery system is substantially in the form of a ring, a sphere, a cylinder, an implant, an intrauterine system, a helical coil, a toroid, a spring, or a combination thereof.

42. The drug delivery system according to claim 41, wherein the system is substantially in the shape of a ring.

43. The drug delivery system according to claim 42, wherein the ring has an outer diameter of from about 50 millimeters to about 60 millimeters, or an outer diameter of from about 52 millimeters to about 56 millimeters.

44. The drug delivery system according to claim 42, wherein the ring has a cross-sectional diameter of from about 2.5 millimeters to about 5 millimeters.

45. The drug delivery system according to claim 1, wherein the steroidal estrogenic compound and the steroidal progestogenic compound are enclosed in separate sections of the core, or enclosed in the same section of the core.

46. The drug delivery system according to claim 1, wherein the steroidal estrogenic compound and the steroidal progestogenic compound are enclosed in same or separate compartments within the core.

47. The drug delivery system according to claim 1, wherein the core includes a fixed ratio of the steroidal estrogenic compound to the steroidal progestogenic compound.

48. The drug delivery system according to claim 1, wherein the subject is a human.

49. A method of contraception comprising:
a) retainably positioning the drug-delivery system of claim 1 within a subject's vaginal tract;
b) retaining the system within the subject's vaginal tract for a period of time;
c) removing the system from the subject after the period of time.

50. The method according to claim 49, wherein the period of time is about 21 days, about 24 days, about 26 days, or about 3 months.

51. A method of contraception for a total period of time, comprising:
(a) retainably positioning the drug-delivery system of claim 1 within a subject's vaginal tract and retaining the system within the subject's vaginal tract for a first period of time;
(b) removing the system from the subject's vaginal tract after the first period of time;
(c) reinserting the system within the subject's vaginal tract after a second period of time and retaining the system within the subject's vaginal tract for a third period of time;
(d) removing the system from the subject's vaginal tract after the third period of time;
(e) optionally reinserting the system within the subject's vaginal tract after the third period of time, retaining the system within the subject's vaginal tract for a fourth period of time, and removing the system from the subject's vaginal tract after the fourth period of time; and
(f) optionally repeating step (e) until the end of the total period of time.

52. The method of claim 51, wherein the first period of time and third period of time are the same amount of time or different amounts of time.

53. The method of claim 51, wherein the second period of time and fourth period of time are the same amount of time or different amounts of time.

54. A method of manufacturing the drug delivery system of claim 1 comprising:
a) producing the core comprising the combination of the steroidal estrogenic compound and the steroidal progestogenic compound, and
b) covering a portion or all of the core with the membrane to form the drug delivery system.

55. The method according to claim 1, wherein the core is coextruded with the membrane to form the drug delivery system.
